(19) United States
(12) Patent Application Publication
(10) Pub. No.: US 2009/0318432 A1
(43) Pub. Date: Dec. 24, 2009

(54) Nitrate esters of aminoalcohols

(75) Inventors: Peter Herold, Munchenstein (CH); Robert Mah, Muttenz (CH); Christiane Marti, Rheinfelden (CH); Nathalie Jotterand, Basel (CH)

Correspondence Address: WENDEROTH, LIND & PONACK, L.L.P., 1030 15th Street, N.W.,, Suite 400 East, Washington, DC, 20005-1503, US

(73) Assignee: SPEEDEL EXPERIMENTA AG, Allschwil (CH)

(21) Appl. No.: 12/310,881

(22) PCT Filed: Sep. 11, 2007
(86) PCT No.: PCT/EP2007/059504

§ 371 Date: Jun. 19, 2009

(30) Foreign Application Priority Data
Sep. 12, 2006 (CH) 01453/06

(51) Int. Cl. A61K 31/538; C07C 229/02; A61K 31/195; A61K 31/445; C07D 211/60; C07D 265/36

(52) U.S. Cl. 514/230.5; 562/451; 514/563; 514/330; 546/245; 544/105

(57) ABSTRACT

The application relates to novel nitrate ester derivatives of substituted aminoalcohols of the general formula (I), wherein R¹, R², R³, V, X, Y and Z₀ have the meanings explained in more detail in the description, to a process for their preparation and to the use of these compounds as therapeutics in cardiovascular diseases, in particular in high blood pressure and vascular and organ damage age accompanying high blood pressure.

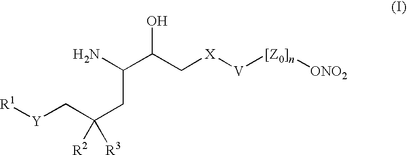

### FIELD OF THE INVENTION

The present invention relates to novel nitrate ester derivatives of substituted amino-alcohols, to methods for their preparation and to their use in cardiovascular diseases, in particular in high blood pressure and vascular and organ damage accompanying high blood pressure. The derivatization of aminoalcohol-based renin inhibitors to give the corresponding nitrate esters leads to compounds having unexpectedly strong hypotensive and tissue-protecting properties, which go beyond the inhibition of renin.

### BACKGROUND OF THE INVENTION

Cardiovascular diseases and further organ damage are the most frequent consequences of untreated or inadequately treated high blood pressure. Thus, high blood pressure in the long term leads to overloading of the heart and vessels and, accompanying this, an increased risk of developing cardiovascular disease and organ damage. Typical consequences are arterial occlusive diseases, cardiac infarct, stroke and kidney damage. In the presence of diabetes or risk factors such as smoking and overweight, the risk of suffering one of these high blood pressure sequelae grows.

In many clinical morbidity and mortality studies, the benefit of an intensive medicinal blood pressure treatment was shown. These studies also showed that therapeutic classes over and beyond lowering of blood pressure can differ in their tissue and organ protection. These distinctions were explained by pleiotropic therapeutic effects. Inhibitors of the renin-angiotensin system, such as angiotensin-converting enzyme (ACE) inhibitors or angiotensin receptor blockers (ARB), were, for example, ascribed anti-inflammatory, anti-proliferative and antithrombotic properties. These properties could be particularly well shown with renin inhibitors and were described, for example, in WO2005/090305.

### OBJECTS OF THE INVENTION

One subject of the invention is therefore substituted aminoalcohols of the general formula

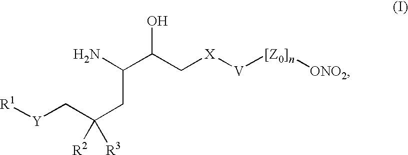

wherein
R¹ is aryl or heterocyclyl, in particular benzoimidazolyl, benzo[1,3]dioxolyl, benzo-furanyl, benzooxazolyl, benzothiazolyl, benzo[b]thienyl, quinazolinyl, quinolyl, quinoxalinyl, 2H-chromenyl, dihydro-2H-benzo[1,4]oxazinyl, dihydro-3H-benzo-[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5-a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetrahydro-imidazo[1,5-a]pyridyl, tetrahydroisoquinolyl, [1,2,3]triazolo[1,5-a]pyridyl or [1,2,4]triazolo[4,3-a]pyridyl, which are substituted by 14 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy-C₁₋₈ alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkyl-heterocyclyl, C₁₋₈-alkoxyaminocarbonyl-C₁₋₈-alkoxy, C₁₋₈-alkoxyaminocarbonyl-C₁₋₈ alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonyl-amino-C₁₋₈-alkyl, C₁₋₈-alkylamidinyl, C₁₋₈-alkylamino-C₁₋₈-alkoxy, di-C₁₋₈-alkylamino-C₁₋₈alkoxy, C₁₋₈-alkylamino-C₁₋₈-alkyl, di-C₁₋₈-alkylamino-C₁₋₈-alkyl, C₁₋₈-alkyl-aminocarbonyl-C₁₋₈-alkoxy, di-C₁₋₈-alkylaminocarbonyl-C₁₋₈-alkoxy, C₁₋₈-alkyl-aminocarbonyl-C₁₋₈alkoxy-C₁₋₈-alkyl, C₁₋₈-alkylaminocarbonyl-C₁₋₈-alkyl, di-C₁₋₈ alkylaminocarbonyl-C₁₋₈-alkyl, C₁₋₈-alkylaminocarbonylamino-C₁₋₈-alkoxy, C₁₋₈-alkyl-aminocarbonylamino-C₁-alkyl, C₁₋₈-alkylcarbonylamino, C₁₋₈-alkylcarbonylamino-C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkyl-carbonyloxy-C₁₋₈-alkyl, C₁₋₈-alkylsulphonyl, C₁₋₈-alkylsulphonyl-C₁₋₈-alkoxy, C₁₋₈-alkyl-sulphonyl-C₁₋₈-alkyl, C₁₋₈-alkylsulphonylamino-C₁₋₈-alkoxy, C₁₋₈-alkylsulphonylamino-C₁₋₈alkyl, optionally N-mono- or N,N-di-C₁₋₈-alkylated amino, aryl-C₁₋₈-alkoxy, aryl-C₁₋₈-alkyl, optionally N-mono- or N,N-di-C₁₋₈-alkylated carbamoyl-C₀₋₈-alkoxy, optionally N-mono- or N,N-di-C₁₋₈-alkylated carbamoyl-C₀₋₈-alkyl, carboxy-C₁₋₈-alkoxy, carboxy-C₁₋₈-alkoxy-C₁₋alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonyl-amino-C₁₋₈-alkoxy, C₃₋₈-cycloalkylcarbonylamino-C₁₋₈-alkyl, O,N-dimethylhydroxyl-amino-C₁₋₈alkyl, halogen, halo-C₁₋₈-alkoxy, halo-C₁₋₈-alkyl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo;
R² and R³, independently of one another, are hydrogen or C₁₋₈-alkyl or both radicals, together with the carbon atom to which they are bonded, are Cm-cycloalkyl;
R⁴ is hydrogen or C₁₋₈-alkyl;

### V is

### -Alk-,

### -Alk-O-Alk-,

-aryl-,

### -Alk-cycloalkyl-,

-cycloalkyl-,
-cycloalkyl-Alk-,

### -Alk-heterocyclyl-,

-heterocyclyl-,
-heterocyclyl-Alk-,

### -Alk-heterocyclyl-C(O)-Alk-, or

-heterocyclyl-C(O)-Alk-;
X is —NR⁴—C(O)— or -Alk-C(O)—NR⁴—, wherein Alk designates C₁₋₈-alkylene;
Y is a bond, —C(O) or —C(O)—NR⁴—;
Z₀ is equal to -Z₁-U-,
wherein Z₀ is —O—C(O)— or —C(O)O—;
U is a bivalent radical having the following meaning:
a)
   C₁₋₈-alkylene, preferably C₁₋₈-alkylene, being optionally substituted with one or more of the substituents selected from the group consisting of: halogen, hydroxyl, —ONO₂ or Tₒ, wherein Tₒ is —OC(O)—(C₁₋₈-alkyl)-ONO₂ or —O—(C₁₋₈-alkyl)-ONO₂;
   C₃₋₈-cycloalkylene, the ring being optionally substituted with side chains T, wherein T is C₁₋₈-alkyl;

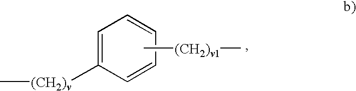

wherein v is an integer from 0 to 20, and v1 is an integer from 1 to 20;

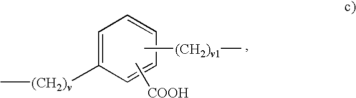

wherein v is an integer from 0 to 20, and v1 is an integer from 1 to 20;

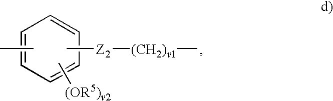

wherein:
v1 is as defined above and v2 is an integer from 0 to 2;
Z₂=—O—C(O)— or —C(O)—O— and R⁵ is H or CH₃;

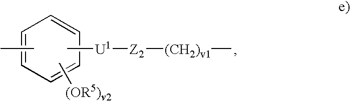

wherein:
v1, v2, R⁵ and Z₂ are as defined above;
U¹ is —CH₂—CH₂— or —CH═CH—(CH₂)ᵥ₂—;

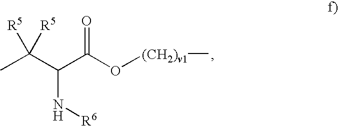

wherein:
v1 and R⁵ are as defined above, R⁶ is H or —C(O)CH₃;

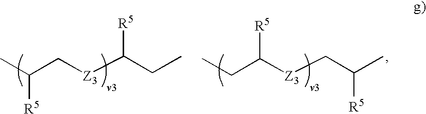

wherein Z₃ is —O— or —S—, v3 is an integer from 1 to 6, preferably from 1 to 4, R⁵ is as defined above; or

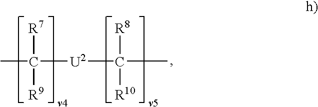

wherein:
v4 is an integer from 0 to 10;
v5 is an integer from 1 to 10;
R⁷, R⁸, R⁹, R¹⁰ are the same or different, and are H or C₁₋₄ alkyl;
U² is a heterocyclic saturated, unsaturated or aromatic 5 or 6 membered ring, containing one or more heteroatoms selected from nitrogen, oxygen and sulphur,
and is preferably selected from

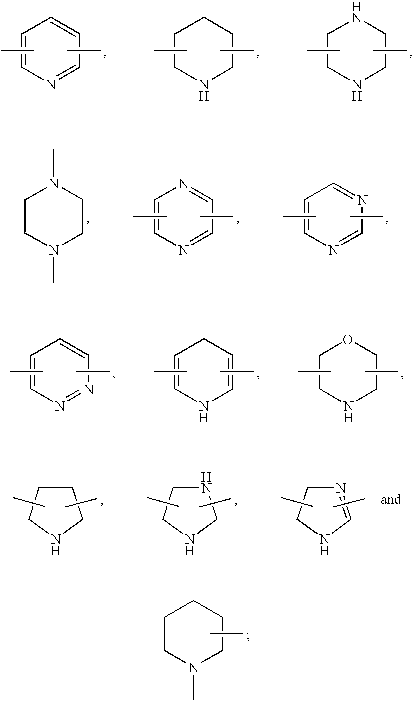

n is 0 or 1;
and their salts, preferably their pharmaceutically usable salts.

The linkage of the above (and hereinafter) mentioned substituent -V- within the compound of the formula (I) starts from —X— with the substituent -V- being arranged from left to right when written as indicated above. For example, the fragment “—X-V-[Z₀]ₙ-ONO₂” of the compound of the formula (I) with V meaning “-heterocyclyl-Alk-” is: “—X-heterocyclyl-Alk-[Z₀]ₙ-ONO₂”.

The linkage of the above (and hereinafter) mentioned substituent —X— within the compound of the formula (I) starts from the alkyl backbone with the substituent —X-being arranged from left to right when written as indicated above. For example, the fragment “—X-V” of the compound of the formula (I) with X meaning “—NR⁵—C(O)—” is: “—NR⁵—C(O)-V”.

The linkage of the above (and hereinafter) mentioned substituent —Y— within the compound of the formula (I) starts from R¹— with the substituent —Y— being arranged from left to right when written as indicated above. For example, the fragment “R¹—Y—” of the compound of the formula (I) with Y meaning “—C(O)—NR⁵—” is: “R¹—C(O)—NR⁵—”.

The linkage of the above (and hereinafter) mentioned substituent -Z₁-U- within the compound of the formula (I) starts from -V- with the substituent -Z₁-U- being arranged from left to right when written as indicated above. For example, the fragment “-V-Z₁-U-ONO₂” of the compound of the formula (I) with -Z₁-U- with Z₁=“—O—C(O)—” and U=“C₁₋₈-alkylene” is: “-V-O—C(O)—C₁₋₈-alkyl-U-ONO₂”.

If not further specified, C₁₋₈-alkyl and alkoxy radicals can be linear or branched. Examples of C₁₋₈-alkyl and alkoxy radicals are methyl, ethyl, n-propyl, isopropyl, n-butyl, isobutyl, sec-butyl, tert-butyl, pentyl, hexyl and methoxy, ethoxy, propoxy, isopropoxy, butoxy, isobutoxy, sec-butoxy and tert-butoxy. C₁₋₈-Alkylenedioxy radicals are preferably methylenedioxy, ethylenedioxy and propylenedioxy. Examples of C₁₋₈-alkanoyl radicals are acetyl, propionyl and butyryl. Cycloalkyl is a saturated, cyclic hydrocarbon radical with 3 up to 12 hydrocarbon atoms, for example cyclopropyl, cyclobutyl, cyclopentyl, cyclohexyl, cycloheptyl, bicyclo[2.2.1]heptyl, cyclooctyl, bicyclo[2.2.2]octyl and adamantyl. Cycloalkyl can be unsubstituted or mono- or polysubstituted, e.g. mono- or disubstituted, by C₁₋₈-alkanoyl, C₂₋₈-alkenyl, C₂₋₈-alkynyl, C₁₋₈-alkoxy, C₁₋₈-alkoxy-C₁₋₈-alkoxy, C₁₋₈-alkoxy-C₁₋₈-alkyl, C₁₋₈-alkoxy-carbonylamino, C₁₋₈-alkyl, C₀₋₈-alkylcarbonylamino, C₁₋₈-alkylcarbonyloxy, C₁₋₈-alkylenedioxy, optionally N-mono- or N,N-di-C₁₋₈-alkylated amino, aryl, optionally N-mono- or N,N-di-C₁₋₈-alkylated carbamoyl, optionally esterified carboxyl, cyano, C₃₋₈-cycloalkoxy, halogen, heterocyclyl, hydroxyl, oxo, polyhalo-C₁₋₈-alkoxy or polyhalo-C₁₋₈-alkyl. C₁₋₈-Alkylene radicals can be linear or branched and are, for example, methylene, ethylene, 1-methylmethylene, propylene, 1-methylethylene, 1-ethyl-methylene, 1,1-dimethylmethylene, 2-methylpropylene, 2-methylbutylene, 2-methyl-propyl-2-ene, butyl-2-ene, butyl-3-ene, propyl-2-ene, tetra-, penta- and hexa-methylene; C₂₋₈-alkenylene radicals are, for example, vinylene and propenylene; C₂₋₈-alkynylene radicals are, for example, ethynylene; acyl radicals are alkanoyl radicals, preferably C₁₋₈-alkanoyl radicals, or aroyl radicals, such as benzoyl. Aryl designates mono- or polynuclear aromatic radicals, which can be mono- or polysubstituted, such as, for example, phenyl, substituted phenyl, naphthyl or substituted naphthyl. Examples of substituents on aryl radicals of this type are acetamidinyl-C₁₋₈-alkyl, acyl-C₁₋₈-alkoxy-C₁₋₈-alkyl, (N-acyl)-C₁₋₈-alkoxy-C₁₋₈-alkyl-amino, C₂₋₄-alkenyl, C₂₋₈-alkenyloxy, C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylamino-carbonyl-C₁₋₈-alkoxy, (N—C₁₋₈-alkoxy)-C₁₋₈-alkylaminocarbonyl-C₁₋₈-alkyl, C₁₋₈-alkoxy-C₁-alkylcarbamoyl, C₁₋₈-alkoxy-C₁₋₈-alkylcarbonyl, C₁₋₈-alkoxy-C₁₋₈-alkyl-carbonylamino, C₁₋₈-alkoxy-C₁₋₈-alkylheterocyclyl, 2-C₁₋₈-alkoxy-C₁₋₈-alkyl-4-oxo-imidazol-1-yl, 6-alkoxyaminocarbonyl-C₁₋₈-alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₅-alkoxycarbonylphenyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxy-carbonylamino, (N—C₁₋₈-alkyl)-C₀₋₈-alkylcarbonylamino-C₁₋₈-alkoxy, (N—C₁₋₈-alkyl)-C₀₋₈-alkylcarbonylamino-C₁₋₈-alkyl, (N—C₁₋₈-alkyl) C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, optionally N-mono- or N,N-di-C₁₋₈-alkylated amino, C₁₋₈-alkylamino-C₂₋₈-alkoxy, di- C₁₋₈-alkylamino-C₂₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈ alkylaminocarbonylamino-C₁₋₈-alkyl, C₁₋₈-alkylcarbamoyl, di-C₁₋₈-alkylcarbamoyl, C₁₋₈-alkylcarbonylamino, C₁₋₈-alkylcarbonylamino-C₁₋₈-alkoxy, C₀₋₈-alkylcarbonylamino-C₁₋₈-alkyl, C₁₋₈-alkylcarbonyloxy, C₁₋alkylcarbonyloxy-C₁₋₈-alkoxy, C₁₋₈-alkylcarbonyloxy-C₁₋₈-alkyl, C₁₋₈-alkylenedioxy, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkyl-sulphonylamino-C₁₋₈-alkyl, aryl-C₁₋₈-alkanoyl, aryl-C₁₋₈-alkoxy, aryl-C₁₋₈-alkyl, arylamino, arylthio, benzoyloxy-C₁₋₈-alkoxy, benzyloxy, carbamoyl, carbamoyl-C₁₋₈-alkoxy, carbamoyloxy-C₁₋₈-alkoxy, carbamoyl-C₁₋₈-alkyl, carboxyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkanoyl, C₃₋₈-cycloalkylcarbonylamino-C₁₋₈-alkoxy, C₃₋₈-cycloalkylcarbonylamino-C₁₋₈-alkyl, cyclopropyl-C-8-alkoxy, cyclopropyl-C₁₋₈-alkyl, O,N-dimethylhydroxylamino-C₁₋₈-alkyl, dioxolanyl-C₁₋₈-alkoxy, halogen, halo-C₁₋₈-alkoxy, halo-C₁₋₈-alkyl, heterocyclyl, heterocyclyl-C₁₋₈-alkanoyl, heterocyclyl-C₁₋₈-alkoxy, heterocyclyl-C₁₋₈-alkoxy-C₁₋₈-alkoxy, heterocyclyl-C₁₋₈-alkoxy-C₁₋₈-alkyl, heterocyclyl-C₁₋₈-alkyl, heterocyclylamino, heterocyclyloxy, heterocyclylthio, hydroxyl, hydroxy-C₁₋₈-alkoxy, hydroxy-C₁₋₈-alkoxy-C₁₋₈-alkoxy, hydroxy-C₁₋₈-alkoxy-C₁₋₈-alkyl, hydroxy-C₁₋₈-alkyl, (N-hydroxy)-C₁₋₈-alkylaminocarbonyl-C₁₋₈-alkoxy, (N-hydroxy)-C₁₋₈-alkylaminocarbonyl-C₁₋₈-alkyl, hydroxy-C₁₋₈-alkylphenyl, (N-hydroxy)aminocarbonyl-C₁₋₈-alkoxy, (N-hydroxy)aminocarbonyl-C₁ alkyl, hydroxybenzyloxy, methylene-dioxybenzyloxy, methoxybenzyloxy, O-methyloximyl-C₁₋₈-alkyl, nitro, 2-oxooxa-zolidinyl-C₁₋₈-alkoxy, 2-oxooxazolidinyl-C₁₋₈-alkyl, phenethoxy, pyridylcarbamoyloxy-C₁₋₈-alkoxy or pyridylcarbonylamino-C₁₋₈-alkyl.

The expression heterocyclyl designates mono- or bicyclic, saturated and unsaturated heterocyclic radicals with 1 to 4 nitrogen and/or 1 or 2 sulphur or oxygen atoms, which can be mono- or polysubstituted, in particular by (in the case of unsaturated heterocyclyl radicals) oxide or oxo or by substituents such as defined above for aryl radicals, or (in the case of saturated heterocyclyl radicals) can be substituted by alkoxy, alkyl or oxo. Heterocyclyl radicals which contain a nitrogen atom can either be bonded to the remaining molecule via the N atom or via a C atom. Preferred heterocyclic radicals each have 1 nitrogen, oxygen or sulphur atom per ring, 1-2 nitrogen atoms and 1-2 oxygen atoms or 1-2 nitrogen atoms and 1-2 sulphur atoms, where at least one, preferably 1-7 carbon atoms, are present per ring.

Examples of heterocyclyl radicals are benzoimidazolyl- benzo[1,3]dioxolyl, benzo-furanyl, benzooxazolyl, benzothiazolyl, benzo[b]thienyl, quinazolinyl, quinolyl, quinoxalinyl, 2H-chromenyl, dihydro-2H-benzo[1,4]oxazinyl, dihydro-3H-benzo-[1,4]oxazinyl, dihydro-2H-benzo[1,4]thiazinyl, 2,3-dihydroindolyl, dihydro-1H-pyrido-[2,3-b][1,4]oxazinyl, furyl, imidazolyl, imidazo[1,2-a]pyridyl, imidazo[1,5-a]pyridyl, indazolyl, indolyl, isobenzofuranyl, isoquinolyl, [1,5]naphthyridyl, oxazolyl, phthalazinyl, pyranyl, pyrazinyl, pyridyl, pyrimidinyl, 1H-pyrrolizinyl, 1H-pyrrolo[2,3-b]pyridyl, 1H-pyrrolo[2,3-c]pyridyl, 1H-pyrrolo[3,2-b]pyridyl, pyrrolyl, tetrahydro-quinolyl, tetrahydroquinoxalinyl, tetrahydroimidazo[1,2-a]pyridyl, tetrahydro-imidazo[1,5-a]pyridyl, tetrahydroisoquinolyl, thiazolyl, thienyl, [1,2,3]triazolo[1,5-a]pyridyl, [1,2,4]triazolo[4,3-a]pyridyl or triazolyl.

Examples of substituted heterocyclyl radicals are 2,2-dimethyl-3-oxo-4H-benzo[1,4]oxazinyl, 2,2-dimethyl-3,4-dihydro-2H-benz[1,4]oxazinyl, 2-aryl-2-methyl-3,4-dihydro-2H-benzo[1,4]oxazinyl, 2,2-dimethyl-2H-chromen-6-yl, 2-aryl-2-methyl-2H-chromen-6-yl, 2-oxobenzoimidazolyl, 2-oxodihydrobenzo[d][1,3]oxazinyl, 4-oxo-dihydroimidazolyl, 5-oxo-4H-[1,2,4]triazinyl, 3-oxo-4H-benzo[1,4]thiazinyl, 1,1,3-trioxodihydro-2H-Iλ⁶-benzo[1,4]thiazinyl, 1-oxopyridyl, 2-oxotetrahydrobenzo-[e][1,4]diazepinyl, 2-oxodihydrobenzo[e][1,4]diazepinyl, 1-oxo-3H-isobenzofuranyl, 4-oxo-3H-thieno[2,3-d]pyrimidinyl, 3-oxo-4H-benzo[1,4]oxazinyl, 1,1-dioxodihydro-2H-benzo[1,4]thiazinyl, 2-oxo-1H-pyrido[2,3-b][1,4]oxazinyl, 2-oxobenzooxazolyl, 2-oxo-1,3-dihydroindolyl, 2-oxodihydro-1H-quinazolinyl, nitrobenzothiazolyl, phenyltetrazolyl, phenyloxadiazolyl, phenylpiperidinyl, phenylpiperazinyl, phenylpyrrolidinyl, thienyloxadiazolyl, furanyloxadiazolyl, benzyloxadiazolyl or phenyloxazolyl.

Examples of saturated heterocyclyl radicals are azetidinyl, dioxolanyl, dioxanyl, dithiolanyl, dithianyl, pyrrolidinyl, piperidinyl, piperazinyl, 4-methylpiperazinyl, morpholinyl, thiomorpholinyl, 2-hydroxymethylpyrrolidinyl, 3-hydroxypyrrolidinyl, 3,4-dihydroxypyrrolidinyl, 4-hydroxypiperidinyl, 4-oxopiperidinyl, 3,5-dimethylmorpholinyl, 4,4-dioxothiomorpholinyl, 4-oxothiomorpholinyl, 2,6-dimethylmorpholinyl, tetrahydro-pyranyl, 2-oxoimidazolidinyl, 2-oxooxazolidinyl, 2-oxopiperidinyl, 2-oxopyrrolidinyl, 2-oxo[1,3]oxazinyl, 2-oxoazepanyl, 2-oxotetrahydropyrimidinyl and the -like.

Examples of bi- or polycyclic heterocyclyl radicals are 2,5-dioxabicyclo[4.1.0]-heptanyl, 2-oxabicyclo[2.2.1]heptanyl, 2-oxabicyclo[4.1.0]heptanyl, 3-oxabicyclo-[4.1.0]heptanyl, 7-oxabicyclo[2.2.1]heptanyl, 2-oxabicyclo[3.1.0]hexanyl, 3-oxa-bicyclo[3.1.0]hexanyl, 1-oxaspiro[2.5]octanyl, 6-oxaspiro[2.5]octanyl, 3-oxa-bicyclo[3.3.1]nonanyl, 2-oxo-1a,7b-dihydro-1H-cyclopropa[c]chromenyl or 1,1a,2,7b-tetrahydrocyclopropa[c]chromenyl.

The expression polyhydroxyalkyl designates C₁₋₈-alkyl radicals which can be substituted with 2-8 hydroxyl groups, such as, for example, glyceryl, arabityl, sorbityl etc.

The expression halogen or halo designates, for example, fluorine, chlorine or bromine, or a radical which is monosubstituted or polysubstituted by fluorine, chlorine or bromine.

The compounds of the formula (I) have at least two asymmetric carbon atoms and can therefore be present in the form of optically pure diastereomers, diastereomer mixtures, diastereomeric racemates, mixtures of diastereomeric racemates or as meso compounds. The invention comprises all these forms. Diastereomer mixtures, diastereomeric racemates or mixtures of diastereomeric racemates can be separated according to customary methods, e.g. by column chromatography, thin-layer chromatography, HPLC and the like.

Salts of compounds with salt-forming groups are in particular acid addition salts, salts with bases or, in the presence of a number of salt-forming groups, optionally also mixed salts or internal salts.

Salts are primarily the pharmaceutically usable or non-toxic salts of compounds of the formula (I).

Salts of this type are formed, for example, from compounds of the formula (I) with an acidic group, e.g. a carboxyl or sulpho group, and are, for example, their salts with suitable bases, such as non-toxic metal-salts derived from metals of group Ia, Ib, IIa and IIb of the periodic table of the elements, e.g. alkali metal salts, in particular lithium, sodium or potassium salts, alkaline earth metal salts, for example magnesium or calcium salts, furthermore zinc salts or -ammonium salts, also those salts which are formed with organic amines, such as mono-, di- or trialkylamines optionally substituted by hydroxyl, in particular mono-, di- or tri-lower alkylamines, or with quaternary ammonium bases, e.g. methyl-, ethyl-, diethyl- or triethylamine, mono-, bis- or tris(2-hydroxy-lower alkyl)amines, such as ethanol-, diethanol- or triethanolamine, tris(hydroxymethyl)methylamine or 2-hydroxy-tertiary-butylamine, N,N-di(lower alkyl)-N-(hydroxy-lower alkyl)amine, such as N,N-di-N-dimethyl-N-(2-hydroxyethyl)amine, or N-methyl-D-glucamine, or quaternary ammonium hydroxides, such as tetrabutylammonium hydroxide. The compounds of the formula I with a basic group, e.g. an amino group, can form acid addition salts, e.g. with suitable inorganic acids, e.g. hydrohalic acid, such as hydrochloric acid, hydrobromic acid, sulphuric acid with replacement of one or both protons, phosphoric acid with replacement of one or more protons, e.g. orthophosphoric acid or metaphosphoric acid, or pyrophosphoric acid with replacement of one or more protons, or with organic carboxylic, sulphonic or phosphonic acids or N-substituted sulphamic acids, e.g. acetic acid, propionic acid, glycolic acid, succinic acid, maleic acid, hydroxymaleic acid, methylmaleic acid, fumaric acid, malic acid, tartaric acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid, isonicotinic acid, furthermore amino acids, such as, for example, the α-amino acids mentioned further in advance, and methanesulphonic acid, ethanesulphonic acid, 2-hydroxyethanesulphonic acid, ethane-1,2-disulphonic acid, benzenesulphonic acid, 4-toluenesulphonic acid, naphthalene-2-sulphonic acid, 2- or 3-phosphoglycerate, glucose 6-phosphate, N-cyclohexylsulphamic acid (with formation of the cyclamates) or with other acidic organic compounds, such as ascorbic acid. Compounds of the formula I with acidic and basic groups can also form internal salts.

For isolation and purification, pharmaceutically unsuitable salts can also be used.

The compound groups mentioned below are not to be considered as complete, but in a useful manner, for example for the replacement of general by more specific definitions, parts of these compound groups can mutually be replaced with one another or by the definitions given above or omitted. The definitions mentioned apply in the context of the general chemical principles, such as, for example, the customary valencies for atoms.

Preferred compounds according to the invention are those of the general formula (II)

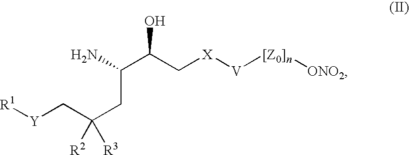

in which R¹, R², R³, V, X, Y and Z₀ have the meaning indicated above for the compounds of the formula (I).

A further, preferred group of compounds of the formula (I), or particularly preferably of the formula (II) and their salts, preferably their pharmaceutically usable salts, are compounds, wherein

R¹ is aryl or heterocyclyl, in particular benzoimidazolyl, benzo[1,3]dioxolyl, benzo-furanyl, benzooxazolyl, benzothiazolyl, benzo[b]thienyl, quinazolinyl, quinolyl, quinoxalinyl, 2H-chromenyl, dihydro-2H-benzo[1,4]oxazinyl, dihydro-3H-benzo[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetrahydroimidazo[1,5-a]pyridyl, tetrahydroisoquinolyl, [1,2,3]triazolo[1,5a]pyridyl or [1,2,4]triazolo[4,3-a]pyridyl, which are substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋alkanoyl, C₁₋alkoxy, C₁₋₈-alkoxy-C₁₋alkanoyl, C₁₋₈-alkoxy, C₁₋₈-alkoxy, C₁₋₈-alkoxy-C₁₋₈-alkoxy-C₁₋₈alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy-C₁₋₈-alkylheterocyclyl, C₁₋₈-alkoxyaminocarbonyl-C₁₋₈-alkoxy, C₁₋₈-alkoxyamino-carbonyl-C₁₋₈alkyl, C₁₋₈-alkoxycarbonyl, C₁₋₈-alkoxycarbonyl-C₁₋₈-alkoxy, C₁₋₈-alkoxy-carbonyl-C₁₋₄alkyl, C₁₋₈-alkoxycarbonylamino-C₁₋₈-alkoxy, C₁₋₈-alkoxycarbonylamino-C₁₋₈alkyl, C₁₋₈-alkyl, (N—C₁₋₈-alkyl)-C₁₋₄alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈ alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkyl-aminocarbonyl-C₁-alkoxy-C₁₋₈-alkyl, C₁₋₈-alkylaminocarbonyl-C₁₋₈-alkyl, di-C₁₋₈ alkylaminocarbonyl-C₁₋₈-alkyl, C₁₋₈-alkylaminocarbonylamino-C₁₋₈-alkoxy, C₁₋₈-alkyl-aminocarbonylamino-C₁₋₈-alkyl, C₁₋₈-alkylcarbonylamino, C₁₋₈-alkylcarbonylamino-C₁₋₈alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkyl-sulphonylamino-C₁₋₈-alkyl, optionally N-mono- or N,N-di-C₁₋₈-alkylated amino, aryl-C₀₋₈-alkoxy, aryl-C₀₋₈-alkyl, optionally N-mono- or N,N-di-C₁₋₄alkylated carbamoyl-C₀₋₈-alkoxy, optionally N-mono- or N,N-di-C₁₋₄-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈alkoxy, cyano-C₁₋₈-alkyl, C₃₋₈cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halo-C₁₋₈-alkoxy, halo-C₁₋₈-alkyl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyl-oximyl-C₁₋₈-alkyl, oxide or oxo;
R² and R³, independently of one another, are hydrogen or C₁₋₈-alkyl or both radicals, together with the carbon atom to which they are bonded, are C₃₋₈-cycloalkyl;
R⁴ is hydrogen or C₁₋₈-alkyl;

### V is

### -Alk-,

### -Alk-O-Alk-,

-aryl-,

### -Alk-cycloalkylene-,

-cycloalkylene-,
-cycloalkylene-Alk-,

### -Alk-heterocyclyl-,

-heterocyclyl-,
-heterocyclyl-Alk-,

### -Alk-heterocyclyl-C(O)-Alk-,

-heterocyclyl-C(O)-Alk-;
X is —NR⁴—C(O)— or -Alk-C(O)—NR⁴—, wherein Alk is C₁₋₈-alkylene;
Y is a bond, —C(O)— or —C(O)—NR⁴—; and

### n is O.

A further, preferred group of compounds of the formula (I), or particularly preferably of the formula (II) and their salts, preferably their pharmaceutically usable salts, are compounds, wherein

R¹ is aryl or heterocyclyl, in particular benzoimidazolyl, benzo[1,3]dioxolyl, benzofuranyl, benzooxazolyl, benzothiazolyl, benzo[b]thienyl, quinazolinyl, quinolyl, quinoxalinyl, 2H-chromenyl, dihydro-2H-benzo[1,4]oxazinyl, dihydro-3H-benzo[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5-a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetrahydroimidazo[1,5-a]pyridyl, tetrahydroisoquinolyl, [1,2,3]triazolo[1,5-a]pyridyl or [1,2,4]triazolo[4,3-a]pyridyl, which are substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈ alkoxy-C₁₋₈-alkoxy, C₁₋₈-alkoxy-C₁₋₈-alkoxy-C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈alkylcarbonylamino, 1-C₁₋₈-alkoxy-C₁₋₈-alkylheterocyclyl, C₁₋₈-alkoxyamino-carbonyl-C₁₋₈alkoxy, C₁₋₈-alkoxyaminocarbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈alkoxy, C₁₋₈-alkoxycarbonylamino-C₁₋₈-alkyl, C₁₋₈-alkyl, (N—C₁₋₈-alkyl)-C₁₋₈-alkoxy-C₁₋₁-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈ alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkyl-amino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkylaminocarbonylamino-C₁₋₈-alkyl, C₁₋₈-alkylcarbonylamino, C₁₋₈-alkylcarbonyl-amino-C₁₋₈-alkoxy, C₁₋₈-alkylcarbonylamino-C₁₋₈-alkyl, C₁₋₄alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonyl-amino-C₁₋₈-alkyl, optionally N-mono- or N,N-di-C₁₋₈-alkylated amino, aryl-C₁₋₈-alkoxy, aryl-C₁₋₈-alkyl, optionally N-mono- or N,N-di-C₁₋₈-alkylated carbamoyl-C₁₋₈-alkoxy, optionally N-mono- or N,N-di-C₁₋₈-alkylated carbamoyl-C₁₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halo-C₁₋₈-alkoxy, halo-C₁₋₈-alkyl, heterocyclyl-C₁₋₈-alkoxy, heterocycyl-C₁₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyloximyl-C₁₋₈-alkyl, oxide or oxo.

Particularly preferably, R¹ is benzoimidazolyl, 2H-chromenyl, 3,4-dihydro-2H-benzo[1,4]oxazinyl, 1a,7b-dihydro-1H-cyclopropa[c]chromenyl, indazolyl, indolyl, 2,3-dihydro-1H-indolyl, phenyl, pyridyl or is 1,1a,2,7b-tetrahydrocyclopropa[c]chromenyl; which are mono- or polysubstituted, as indicated above, very particularly preferably by C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylcarbonylamino-C₁₋₈-alkoxy, C₁₋₈-alkylcarbonylamino-C₁₋₈-alkyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkylsulphonylamino-C₁₋₈-alkyl, C₃₋₈-cycloalkylcarbonylamino-C₁₋₈-alkoxy, C₃₋₈-cycloalkylcarbonylamino-C₁₋₈-alkyl, halogen, halo-C₁₋₈-alkoxy, halo-C₁₋₈-alkyl or oxide.

A further, preferred group of compounds of the formula (I), or particularly preferably of the formula (II) and their salts, preferably their pharmaceutically usable salts, are compounds, wherein

### V is

### -Alk-,

### -Alk-O-Alk-,

### -Alk-cycloalkylene-,

-cycloalkylene-,
-cycloalkylene-Alk-,

### -Alk-heterocyclyl-,

-heterocyclyl-,
-heterocyclyl-Alk-,

### -Alk-heterocyclyl-C(O)-Alk-,

-heterocyclyl-C(O)-Alk-;
where cycloalkyl is a saturated, cyclic hydrocarbon radical with 3 to 8, preferably 3-6 carbon atoms, for example cyclopropyl, cyclobutyl, cyclopentyl, cyclohexyl; which can be unsubstituted or mono- or polysubstituted, e.g. mono- or disubstituted, by C₁₋₈-alkoxy, C₁₋₈-alkyl, optionally N-mono or N,N-di-C₁₋₈-alkylated amino, cyano, halogen, hydroxy or oxo;
where heterocyclyl is preferably a monocyclic, saturated or unsaturated heterocyclic radical with 3 to 8, particularly preferably 3 to 7, ring atoms, among them 1 to 4 nitrogen and/or 1 or 2 sulphur and/or oxygen atoms, for example azepanyl, azetidinyl, aziridinyl, dioxanyl, dioxepanyl, dioxolanyl, dithianyl, dithiolanyl, furanyl, oxathianyl, oxazolidinyl, oxetanyl, oxepanyl, oxiranyl, piperidinyl, pyrrolidinyl, tetrahydrofuranyl, tetrahydropyranyl, tetrahydrothiophenyl, tetrahydrothiopyranyl or thiepanyl which can be unsubstituted or mono- or polysubstituted, e.g. mono-, di-, tri- or tetrasubstituted, in particular by C₁₋₈-alkoxy, C₁₋₈-alkyl, optionally N-mono or N,N-di-C₁₋₈-alkylated amino, aryl, cyano, halogen or hydroxyl.

Particularly preferably, V is

### -Alk-,

-cycloalkyl-,
-heterocyclyl-C(O)-Alk-.

A further, preferred group of compounds of the formula (I), or particularly preferably of the formula (II) and their salts, preferably their pharmaceutically usable salts, are compounds, wherein

Z₀ is equal to -Z₁-U-,
wherein Z₁ is —O—C(O)— or O—C(O)O—;
U is a bivalent radical having the following meaning:
a)
linear or branched C₁₋₈-alkylene;

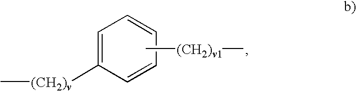

wherein v is 0 or 1, and v1 is 1; or

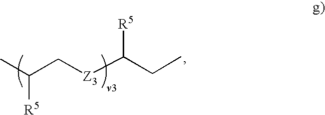

wherein Z₃ is —O— or —S—, v3 is 1 and R⁵ is H.

The compounds of the formulae (I) and (II) can be prepared in an analogous manner to preparation processes known from the literature. Similar preparation processes are described, for example, in EP 678503, WO2005/070870, WO2005/070871, WO2005/070877 and WO2005/090305. Details of the specific preparation variants can be taken from the examples.

The compounds of the formulae (I) and (II) can also be prepared in optically pure form. Separation into antipodes can be carried out by methods known per se either preferably in an early stage in the synthesis by salt formation with an optically active acid such as, for example, (+)- or (−)-mandelic acid and separation of the diastereomeric salts by fractional crystallization or preferably in a rather late stage by derivatization with a chiral auxiliary structural unit such as, for example, (+)- or (−) camphanyl chloride, and separation of the diastereomeric products by chromatography and/or crystallization and subsequent cleavage of the bond to the chiral auxiliary. The pure diastereomeric salts and derivatives can be analysed using customary spectroscopic methods for determination of the absolute configuration of the piperidine contained, X-ray spectroscopy on single crystals being a particularly suitable method.

The compounds of the formulae (I) and (II) also include those compounds in which one or more atoms are replaced by their stable, non-radioactive isotopes; for example a hydrogen atom by deuterium.

Prodrug derivatives of the compounds presently described are derivatives thereof which in in vivo use release the original compound by means of a chemical or physiological process. A prodrug can be reacted, for example, to give the original compound on achieving a physiological pH or by enzymatic conversion. Prodrug derivatives can be, for example, esters of freely available carboxylic acids, S- and O-acyl derivatives of thiols, alcohols or phenols, the acyl group being defined as presently. Pharmaceutically usable ester derivatives, which can be reacted by solvolysis in physiological medium to give the original carboxylic acid, such as, for example, lower alkyl esters, cycloalkyl esters, lower alkenyl esters, benzyl esters, mono- or disubstituted lower alkyl esters, such as lower ω-(amino, mono- or dialkylamino, carboxyl, lower alkoxycarbonyl)alkyl esters or such as lower α-(alkanoyloxy, alkoxycarbonyl or dialkylaminocarbonyl)alkyl esters are preferred; those utilized are conventionally pivaloyloxymethyl esters and similar esters.

On account of the close relationship between a free compound and a salt compound, a certain compound in this invention also comprises its salt form, if this is possible and appropriate.

The compounds of the formula (I) or (II), in particular the compounds illustrated in Examples 1-48 and their pharmaceutically usable salts show extended properties in that on the one hand they release nitrogen monoxide and on the other hand either directly inhibit the natural enzyme renin as illustrated by the Examples 1-36 or inhibit renin indirectly upon drug metabolism as illustrated by the Examples 37-48. The release of nitrogen monoxide and its pharmacokinetic distribution in the living organism resulted in unexpectedly useful pharmacological properties, which can be explained as follows.

The juxtaglomerular cells of the kidney secrete the enzyme renin into the systemic circulation, where renin proteolytically cleaves the substrate angiotensinogen into the decapeptide product angiotensin I. Angiotensin I is further processed proteolytically by the angiotensin-converting enyzme to the octapeptide angiotensin II. Angiotensin II increases the blood pressure by causing arterial vasoconstriction by receptor binding and a decrease in sodium excretion. Moreover, angiotensin II increases the activity of NADPH oxidase and thus leads to the breakdown of nitrogen monoxide, which leads to a decrease in endothelium-dependent vascular relaxation, to hypertrophy, proliferation and migration of the smooth muscle cells, to the formation of extracellular matrix, and to thrombotic and inflammatory reactions.

The inhibition of the enzymatic renin activity leads to decreased formation of angiotensin I and thus to the lowering of the angiotensin II content.

The suppression of angiotensin II formation leads to a decrease in the arterial blood pressure and to the prevention of blood pressure-dependent and blood pressure-independent tissue damage.

Nitrogen monoxide has the property of reversibly activating soluble guanylate cyclase, a widespread enzyme which can be found in the cells of any organ system. In this process, nitrogen monoxide binds to the haem-containing domain group of the enzyme, by means of which its catalytic activity, which converts guanosine triphosphate to cyclic guanosine monophosphate, is increased. Increased concentrations of cyclic guanosine monophosphate relax the smooth muscle cells in the venous and, in some cases, arterial blood vessels, in the heart, in the intestine, in the urogenital tract, in the airways and in the uterus; they moreover inhibit the aggregation and adhesion of platelets and block the accumulation of leucocytes on the vessel walls. These effects, so it is believed, explain the vasculoprotective contributions of nitrogen monoxide. In fact, virtually any cardiovascular risk factor, such as, for example, high blood pressure, diabetes, hyperlipidaemia and smoking can be connected with a decrease in the basal and in the stimulated nitrogen monoxide-induced vascular relaxation.

The invention described herein makes available a new therapeutic approach for high blood pressure and blood pressure-dependent and blood pressure-independent diseases in that the compounds of the formulae (I) and (II) on the one hand inhibit high blood pressure-promoting and tissue-damaging renin and on the other hand release tissue-relaxing and tissue-protecting nitrogen monoxide. Pharmacologically, the compounds of the formulae (I) and (II) allow the relaxation of the arterial and of the venous blood vessels by systemic distribution of the compounds and thus systemic renin inhibition and nitrogen monoxide release. In contrast to the separate use of both active principles, i.e. of a renin inhibitor and of a nitrogen monoxide-releasing substance, the compounds of the invention described herein allow a uniform systemic distribution and thus a balanced effect on blood and blood vessels, blood pressure and tissue.

The pharmacological profile of the compounds of the formulae (I) and (II) can be worked out using the following system. The in vitro test systems described herein allow, independently of one another, the direct determination of the renin-inhibiting properties and the nitrogen monoxide-releasing properties of a compound. The in vivo test systems described herein allow the determination of the combined effect of renin inhibition be it occurring directly or upon drug metabolism and nitrogen monoxide release on the blood pressure and on tissue functions.

In order to distinguish the blood pressure-lowering and tissue-protecting contributions of renin inhibition from nitrogen monoxide release of an orally administered compound, the blood pressure lowering and tissue function of a nitrate ester compound are compared with the nitrate-free parent substance with equal pharmacokinetic distribution. If the release of the nitrogen monoxide does not take place or takes place in a pharmacologically unsuitable manner, no additional blood pressure lowering or no tissue protection can be shown. If, however, the pharma-cokinetic release and distribution, for probably not conclusively explainable reasons, take place on the nitrogen monoxide-sensitive and blood pressure-regulating tissues, as shown presently an additional blood pressure-lowering or tissue protection of the heart and kidneys can be functionally shown.

The determination of the direct renin-inhibiting properties of a compound can be carried out using in vitro enzymatic test systems. The in vitro test systems determine the formation of angiotensin I from natural or recombinant renin substrate in the presence of human plasma samples or purified renin enzyme. A frequently used in vitro test system has been described by Nussberger et al. (1987) in J. Cardiovascular Pharmacology, Vol. 9, pp. 39-44. The test quantitatively determines the formation of angiotensin I in human plasma in the presence or absence of renin inhibitors in various concentration ranges. The angiotensin I concentrations produced are measured by a radioimmuno investigation.

The compounds of the present invention, particularity the compounds illustrated in Examples 1-36, showed in these in vitro test systems direct renin-inhibiting actions in minimal concentration ranges from 10⁻¹⁰ to 10⁻⁷ mol/l. The efficacy of the compounds can be expressed using IC₅₀ values, which represent the concentration of a certain compound which suppresses the angiotensin I content by 50%. The IC₅₀ values of the compounds obtained were in the range from 0.1 nM to 100 nM, the majority of these in the range from 1 nM to 10 nM. Certain nitrate ester compounds showed similar, i.e. higher, identical or alternatively lower, IC₅₀ values in comparison with their nitrate-free parent substances.

The determination of the nitrogen monoxide-releasing properties of a compound, be it by enzymatic or non-enzymatic action, can be carried out by means of in vitro vascular reactivity tests. The in vitro vascular reactivity test measures the ability of the released nitrogen monoxide to relax a pretensioned aorta ring or vein ring which is kept in an organ chamber. Frequently used instructions are described by Gonzales et al., in Adv. Physiol. Educ. (2000) 24:13-21. The thoracic aorta or the femoral vein is isolated from sacrificed and exsanguinated Wistar rats and cut up to give rings 2 mm long. The arterial rings are stored in organ chambers. Changes in the development of tension after the action of compounds are recorded by an isometric signal transmitter, which is connected to a computerized detection system. The computer program analyses time curves, period and size amounts between contractions. The test compounds described here showed vessel-relaxing actions in phenylephrine-precontracted vessels in concentration ranges of approximately 10⁻⁹ to 10⁻⁴ mol/l. The nitrogen monoxide-induced vessel-relaxing activity of the compounds can be expressed in percentage points of the maximal vessel relaxation achieved with sodium nitroprusside (SNP) relative to the phenylephrine (0.1 μM)-induced contractile tone.

The determination of the in vitro inhibition of platelet aggregation (IPA) by nitrogen monoxide can be carried out using the following test system. Platelet-enriched plasma is obtained from rat blood by centrifugation. The aggregability of the platelets can be measured optically using a turbidimetric aggregometer. The aggregation-inhibiting action of the compounds of the formulae (I) and (II) can be determined relative to aggregation-stimulating agents using adenosine diphosphate (ADP). The test compounds (10-500 μM) can be added to the platelet solution here 1-5 minutes before introduction of ADP (1-10 uM). The optical density determined in the test system determines the degree of platelet aggregation.

The compounds of the formulae (I) and (II) led to a decrease in the ADP-induced platelet aggregation. The percentage decrease was between 20 and 60%.

The in vivo blood pressure-lowering actions of the compounds of the formulae (I) and (II) can be shown in doubly transgenic rats (dTGR), which overexpress both the gene for human angiotensinogen and the gene for human renin and as a result of this develop hypertension; Blood pressure values and heart rate values can be determined continuously, for example, by means of chronically implanted telemetry equipment. A telemetry system can consist of a radio frequency transmitter, a receiver apparatus and a data collection system. The pressure transmission catheter of the pressure sensor can be implanted into the abdominal aorta. After the operation, the rats are allowed a recovery period of 7 days, where the telemetry recording should indicate the restoration of a 24 hour oscillation rhythm of blood pressure and heart rate before compounds can be tested.

Compounds of the formulae (I) and (II) can be administered orally by means of stomach tubes. Blood pressure changes can be recorded over 24 hours after a single dosage or continuously over 2 to 42 days after multiple dosage. The dose administered, consisting of vehicle-suspended or dissolved compound, is in the range from 0.5 mg/kg of body weight up to 100 mg/kg of body weight. Blood pressure changes can be expressed by means of mean arterial blood pressure values (MAP), in order to describe the average pressure within a cardiac cycle.

The use of the compounds of the formulae (I) and (II) resulted in lowerings of mean arterial blood pressure (MAP) of 20 to 70 mmHg. Moreover, certain nitrate ester compounds showed greater lowerings of blood pressure in comparison to their nitrate-free parent compounds.

The pharmacokinetic distribution of a compound of the formula (I) or (II) can be determined by comparison of the time-dependent plasma concentrations of a compound after oral or intravenous administration to a living organism such as, for example, a mouse, a rat or a dog. The doses used for oral administration range from 0.5 to 50 mg/kg of body weight; for intravenous administration doses of 0.5 to 20 mg/kg of body weight are administered. For intravenous use, the usable formulation of a compound can be administered to groups of three to eight animals, for example, to the caudal vein of a rat and, for oral use, by means of a stomach tube. Ethically justifiable blood sample volumes can be taken from the animals, according to a suitable time pattern, for example automatically by means of an AccuSampler (DiLab Europe, Lund, Sweden), and transferred to heparinized containers. Plasma samples produced are stored, up to the determination of the concentration of a compound contained, for example by liquid chromatography and mass spectral analysis, at −17 to −23° C. Compounds of the formulae (I) and (II) showed absolute bioavailabilities in the range from 10-50% and elimination half-lives of 2-12 h. The plasma levels of a compound can also be expressed by area under the curve (AUC) values, which allow an additional comparison of the compounds. Thus, compounds of the formulae (I) and (II) show AUC values in the range from 500 to 15 000 ng×h/ml.

The influencing of the coronary flow and anti-ischaemic effects of the compounds of the formulae (I) and (II) can also be measured in vivo by means of a perfusion model. For this, the heart is removed from the rats pretreated with the compounds of the formulae (I) and (II) after anaesthesia and this is mounted in a Langendorff apparatus after cannulation of the aorta, whereby it can be supplied with oxygen-rich perfusate. After tying off the vena cava and pulmonary veins, and the cannulation of the pulmonary artery, the coronary flow can be determined by means of a flow meter. The coronary flow was measured volumetrically and expressed as the millilitres of perfusate which are collected in one minute. The coronary flow can be measured before and after induction of ischaemia, ischaemia being initiated by interruption of the perfusion for 30 minutes and a 30-minute reperfusion period following after opening of the flow. Thus the coronary flow in the reperfusion period can be compared with that in the pre-ischaemic period. Compounds of the formulae (I) and (II) are thereby able to increase the coronary flow by 50-200%.

The tissue-protecting action of a compound of the formula (I) and (II) after long-term use can be determined in vivo by proteinuria and kidney function measurements, as indicators of kidney damage. The investigations can be carried out in 4-week-old, male rats, for human renin and angiotensinogen doubly transgenic rats (dTGR). The animals are divided into treatment groups and are given drug, comparison substance or vehicle (control) for 7 weeks. The dose used for oral administration can range from 0.5 to 100 mg/kg of body weight. During the entire study period, the animals are given standard feed and tap water ad libitum. The animals are placed in a metabolic cage once weekly in order to determine the 24-hour albumin excretion (AE) in the urine, diuresis, natriuresis and urine osmolality. Moreover, the kidneys can be functionally investigated by determining the glomerular filtration rate, for example using the iohexyl method, creatinine excretion, for example by means of the plasma creatinine concentration, and the renal perfusion rate, for example using the para-aminohippurate method. At the end of the study, the animals are sacrificed and the kidneys and hearts are removed for weight determination and immunohistological investigations (tissue fibrosis, leucocyte infiltration, inflammation markers etc.). The extent of tissue fibrosis can be shown, for example, with the following polyclonal antibodies: anti-fibronectin, anti-collagen IV. The extent of cell infiltration can be shown, for example, with the following monoclonal antibodies: anti-CD4, anti-CD8, anti-ED1, anti-MHCII, anti-CD68 and anti-Ox6. As inflammation markers, immunologically captured TGFβ, MCP-1, TNFα or IL-6 can be used. The semiquantitative evaluation of various kidney and heart sections showed a decrease in tissue fibrosis and tissue inflammation after use of the compounds of the formulae (I) and (II).

| | IC₅₀ | IPA | MAP | | AE | CF | |
|---|---|---|---|---|---|---|---|
| | [μM] | [%] | [mm Hg] | AUC | [μg/24 h] | [%] | Fib Cl |
| Compound X | 10-100 | 50 | 20-70 | 500-15 000 | 100-5000 | 50-200 | + |
| Parent | 0 | 20 | 20-60 | 500-15 000 | 200-10 000 | 20-50 | ++ |
| compound X | | | | | | | |

The comparison of these pharmacological parameters allows not only the improved profile of a nitrate ester compound to be observed compared to its nitrate-free parent compound, but also the optimal nitrate ester compounds of this invention to be identified. Abbreviations: IC, inhibitory concentration; pIC, negativer logarithm of the IC; IPA, inhibition of platelet aggregation; MAP, mean arterial pressure; AUC, area under the curve; AE, albumin excretion; CF, coronary flow; Fib, fibrosis; CI, cell infiltration.

In order to achieve the desired effects in a patient to be treated, the compounds of the present invention can be administered orally or enterally, such as, for example, intravenously, intraperitoneally, intramuscularly, rectally, subcutaneously or alternatively by direct injection of the active agent locally into tissue or tumours. The term patient paraphrases warm-blooded animals and mammals, such as, for example, humans, primates, cattle, dogs, cats, horses, sheep, mice, rats and pigs. The compounds can be administered as a pharmaceutical preparation or incorporated into an administration device which guarantees lasting effusion of the compound. The amount of substance to be administered can vary over a wide range and can be any effective dose. Depending on the patient to be treated or condition to be treated and type of administration, the dose of the effective active agent can be between approximately 0.005 and 50 milligrams per kilogram of body weight daily, but preferably between approximately 0.05 and 15 milligrams per kilogram of body weight daily.

For oral administration, the compounds can be formulated in solid or liquid pharmaceutical forms such as, for example, as capsules, pills, tablets, coated tablets, granules, powders, solutions, suspensions or emulsions. The dose of a solid pharmaceutical form can be an ordinary hard gelatin capsule, which can be filled with active agents and excipients, such as lubricants and fillers, such as, for example, lactose, sucrose and maize starch. A further administration form can be the tabletting of the active substance of the present invention. The tabletting can be carried out with conventional tabletting auxiliaries such as, for example, lactose, sucrose, maize starch, combined with binders consisting of acacia gum, maize starch or gelatin, disintegrants such as potato starch or cross-linked polyvinylpyrrolidone (PVP) and lubricants such as stearic acid or magnesium stearate. Suitable excipients for soft gelatin capsules are, for example, vegetable oils, waxes, fats, semisolid and liquid polyols etc. Suitable excipients for the preparation of solutions and syrups are, for example, water, polyols, sucrose, invert sugar, glucose etc.

For rectal administration, the compounds can be formulated in solid or liquid pharmaceutical forms such as, for example, suppositories. Suitable excipients for suppositories are, for example, natural or hardened oils, waxes, fats, semiliquid or liquid polyols etc.

For parenteral administration, the compounds can be formulated as an injectable dose of the active agent in a liquid or suspension. The preparations usually contain a physiologically tolerable sterile solvent, which can contain a water-in-oil emulsion, with or without surfactant, and other pharmaceutically acceptable excipients. Oils which can be used for preparations of this type are paraffins and triglycerides of plant, animal or synthetic origin, such as, for example peanut oil, soya bean oil and mineral oil. In general, injectable solutions contain liquid carriers such as more preferably water, common salt, dextrose or related sugar solutions, ethanol and glycols, such as propylene glycol or polyethylene glycol.

The substances can be administered as a transdermal patch system, as a depot injection or implant if the formulation makes possible a lasting release of the active agent. The active agent can be compressed as granules or to give narrow cylinders and can be administered subcutaneously or intramuscularly as a depot injection or implant.

In addition, the pharmaceutical preparations can further contain preservatives, solubilizers, viscosity-increasing substances, stabilizers, wetting agents, emulsifiers, sweeteners, colourants, flavourings, salts for alteration of the osmotic pressure, buffers, coating agents or antioxidants. They can also contain other therapeutically valuable substances.

A further subject of the present invention is the use of the compounds of the formula (I), or preferably of the formula (II), and their pharmaceutically usable salts in the treatment and/or prevention of renin-mediated diseases, the severity of which is increased by nitrogen monoxide deficiency, such as, for example, high blood pressure, left-ventricular hypertrophy, heart failure, stable angina pectoris, unstable angina pectoris, angina, acute coronary syndrome, vasospastic angina, stroke, ischaemic disorders, cardiac infarct, ischaemic reperfusion injuries, Raynaud's syndrome, thrombosis, atrial fibrillation, cardiac arrhythmias, dyslipidaemia, atherosclerosis, prevention of stenoses after angioplasties, peripheral arterial occlusive diseases, erectile disorders, diabetes type 1 and type 2, diabetic complications, nephropathy, retinopathy, neuropathy, pulmonary arterial hyper-tension, disorders of the digestive tract, portal hypertension, cirrhosis of the liver.

The presently described compounds of the invention allow the following methods of use:
As a therapeutic combination in the form of a preparation or of a kit which is composed of individual components, consisting of a presently described compound, in free form or as a pharmaceutically usable salt, and at least one pharmaceutical form whose active agent has a blood pressure-lowering, an inotropic, an antidiabetic, a lipid-lowering or an antioxidizing action, which can be used either simultaneously or sequentially. The preparation and the kit can contain use instructions.
As a method for combined use, such as, for example, in a simultaneous or sequential sequence, of a therapeutically efficacious amount of a compound described here, in free or in pharmaceutically usable salt form, and of a second active agent having blood pressure-lowering, inotropic, anti-diabetic, lipid-lowering or anti-oxidizing action.

The presently described compounds and their pharmaceutically usable salts can be used in combination with:
(i) one or more blood pressure-lowering active agents, as of the type, for example:
   angiotensin II receptor inhibitors such as candesartan, irbesartan, olmesartan, losartan, valsartan, telmisartan, eprosartan etc.;
   angiotensin-converting enzyme (ACE) inhibitors such as quinopril, ramipril, trandolapril, lisinopril, captopril, enalapril etc.;
   calcium channel inhibitors such as nifedipine, nicardipine, verapamil, isradipine, nimodipine, amlodipine, felodipine, nisoldipine, diltiazem, fendiline, flunarizine, perhexyline, gallopamil etc.;
   diuretics such as hydrochlorothiazide, chlorothiazide, acetazolamide, amiloride, bumetanide, benzthiazide, ethacrynic acid, furosemide, indacrinone, metolazone, triamterene, chlorthalidone etc.;
   aldosterone receptor antagonists such as spironolactone, eplerenone;
   endothelin receptor antagonists such as bosentan;
   phosphodiesterase inhibitors such as aminone, sildenafil;
   direct vasodilators such as dihydralazine, minoxidil, pinacidil, diazoxide, flosequinan etc.;
   α- and β-adrenergic receptor antagonists such as phentolamine, phenoxybenzamine, prazosine, doxazosine, terazosine, carvedilol, atenolol, metoprolol, nadolol, propranolol, timolol, carteolol etc.;
   neutral endopeptidase (NEP) inhibitors;
   sympatholytics such as methyldopa, clonidine, guanabenz, reserpine;
(ii) one or more inotropic active agents, as of the type, for example:
   cardiac glycosides such as digoxin;
   β-adrenergic receptor stimulators such as dobutamine
   thyroid hormones such as thyroxine
(iii) one or more anti-diabetic active agents, as of the type, for example:
   insulins such as insulin aspartate, human insulin, insulin lispro, insulin glargine and other rapid-, medium- or long-acting insulin derivatives and combinations;
   insulin sensitizers such as rosiglitazone, pioglitazone;
   sulphonylureas such as glimepiride, chlorpropamide, glipizide, glyburide etc.;
   biguanides such as metformin;
   glucosidase inhibitors such as acarbose, miglitol,
   meglitinides such as repaglinide; nateglinide etc.;
(iv) one or more lipid-lowering active agents, as of the type, for example:
   HMG-CoA reductase inhibitors such as lovastatin, fluvastatin, pravastatin, atorvastatin, simvastatin, rosuvastatin etc.;
   fibrate derivatives such as fenofibrate, gemfibrozil etc.;
   bile acid-binding active agents such as colestipol, colestyramine, colesevelam;
   cholesterol absorption inhibitors such as ezetimibe;
   lipase inhibitors such as odistate;
   nicotinic acid such as niacin
(v) one or more active agents with a direct or indirect anti-oxidant effect, as of the type, for example:
   vitamins and vitamin derivatives such as beta-carotene, lycopene, vitamin A such as retinol (vitamin A1), 3,4-didehydroretinol (vitamin A2), and 3-hydroxyretinol (vitamin A3), vitamin C or ascorbic acid and vitamin E or α-tocopherol,
   lipoic acid, 2-mercaptoethane sulphonate, cysteine,
   enzymes such as superoxide dismutase, catalase etc.;
      and other active agents which are used for the treatment of high blood pressure, vascular, kidney and liver disorders and are also usable for the prevention of medicinal tolerance symptoms. Combinations of this type can be used separately or in preparations which contain a number of components.

The presently described compounds and their pharmaceutically usable salts can be of use as combinations with:
(i) a diagnostic test system which allows the quantitative determination of the plasma renin concentration (PRC),
(ii) a diagnostic test system which allows the quantitative determination of the plasma aldosterone concentration (PAC),
(iii) a diagnostic test system which allows the quantitative determination of the plasma renin activity (PRA),
(iv) a diagnostic test system which allows the quantitative determination of the ratio of the plasma aldosterone concentration to the renin concentration (ARC),
(v) a diagnostic test system which allows the quantitative determination of the ratio of the plasma aldosterone concentration to the plasma renin activity (ARR).

Such combinations of a diagnostic test system and a therapy can be used independently or as a preparation with individual components.

### EXAMPLES

The following examples illustrate the present invention. All temperatures are indicated in degrees Celsius, pressures in mbar. If not mentioned otherwise, the reactions take place at room temperature. The abbreviation “Rf=xx (A)” means, for example, that the Rf value xx is determined in the solvent system A. The quantitative ratio of solvents to one another is always indicated in parts by volume. Chemical names for final products and intermediates were generated with the aid of the program AutoNom 2000 (Automatic Nomenclature).

Thin layer chromatography mobile phase systems:

A dichloromethane-methanol-ammonia conc. 25%=200:20:1
B dichloromethane-methanol-ammonia conc. 25%=200:20:0.5
C dichloromethane-methanol-ammonia conc. 25%=200:10:1
D dichloromethane-methanol-ammonia conc. 25%=90:10:1
E dichloromethane-methanol-ammonia conc. 25%=60:10:1
F dichloromethane-methanol-ammonia conc. 25%=200:30:1
G dichloromethane-methanol=9:1

HPLC gradient on Hypersil BDS C-18 (5 μm); column: 4×125 mm
I 90% water*/10% acetonitrile* to 0% water*/100% acetonitrile* in 5 minutes+2.5 minutes (1.5 ml/min)
H 95% water*/5% acetonitrile* to 0% water*/100% acetonitrile* in 40 minutes (0.8 ml/min)
*contains 0.1% trifluoroacetic acid

The following abbreviations are used:
Rf ratio of running distance of a substance to distance of the eluent front from the starting point in thin layer chromatography
Rt retention time of a substance in HPLC (in minutes)
M.p. Melting point (temperature)

### General Method Q (N-Boc Deprotection II)

15 mmol of trifluoroacetic acid are added dropwise at 0° C. to a solution of 1 mmol of “N-Boc derivative” in 10 ml of dichloromethane. The reaction mixture is stirred for 2 hours at room temperature. Subsequently, it is poured onto 15 ml of ice-cooled, saturated sodium hydrogencarbonate solution and the mixture is extracted three times with 30 ml each of ethyl acetate. The combined organic phases are dried using sodium sulphate and evaporated. The title compound is obtained from the residue by means of flash chromatography (SiO₂ F60).

### General Method R(N-Boc Protection)

1.15 mmol of di-tert-butyl dicarbonate are added to a solution of 1 mmol of “amine” and 1.2 mmol of triethylamine in 12 ml of dichloromethane. The reaction mixture is stirred for 14 hours at room temperature. Subsequently, it is diluted with 20 ml of dichloromethane and the organic phase is extracted successively with 10 ml of 0.2N HCl, 10 ml of saturated sodium hydrogencarbonate solution and 10 ml of brine. The organic phase is dried using sodium sulphate and evaporated. The title compound is obtained from the residue by means of flash chromatography (SiO₂ F60).

### General Method S (Mesylate Nitration)

1.6 mmol of tetrabutylammonium nitrate are added to a solution of 1 mmol of “mesylate” in 5 ml of toluene. The reaction mixture is stirred for 18 hours at 110° C. Subsequently, it is filtered through silica gel (eluent EtOAc-heptane 1:1) and the resulting solution is evaporated.

### General Method U (Amide Coupling)

5.0 mmol of triethylamine and 1.0 mmol of tripropylphosphonic cyclic anhydride [68957-94-8] (50% in ethyl acetate) are added at room temperature to a solution of 1.0 mmol of “acid” and 1.0 mmol of “amine” in 20 ml of dichloromethane. The reaction mixture is stirred for 1-3 hours, diluted with dichloromethane and washed with 1 N HCl and brine. The combined organic phases are dried using sodium sulphate and evaporated. The title compound is obtained from the residue by means of flash chromatography (SiO₂ F60).

### General Method V: (Lactone Amidation)

A mixture of 1 mmol of “lactone”, “amine” (5-30 equiv.) and 1 mmol of 2-hydroxy-pyridine is stirred for 2-72 hours at 40-55° C. The reaction mixture is treated with 30 ml of 1 M sodium hydrogencarbonate solution and extracted with tert-butyl methyl ether (2×). The combined organic phases are dried using sodium sulphate, filtered and the filtrate is evaporated. The title compound is obtained from the residue by means of flash chromatography (SiO₂ 60F).

### General Method W: (Grignard Reaction)

A solution of 1 mmol of dibutylmagnesium (1 M in heptane) in 3.6 ml of tetrahydrofuran is cooled to 0° C. and treated dropwise at 0° C. with 1 mmol butyllithium solution (1.6M in hexane). The mixture is stirred for 10 minutes at 0° C. A solution of 1 mmol of “aryl bromide” or “heteroaryl bromide” in 1.4 ml of tetrahydrofuran is added dropwise at 0° C. The reaction mixture is stirred for 15 minutes at 0° C., cooled to −78° C. and a solution of 1 mmol of 2-[2-azido-2-(4-isopropyl-5-oxotetrahydrofuran-2-yl)ethyl]-3-methylbutyraldehyde [173154-024] in 1.4 ml of tetrahydrofuran is added dropwise at −78° C. The reaction mixture is stirred for 1 hour at −78° C. and quenched with 1M ammonium chloride solution. It is extracted with tert-butyl methyl ether (3×). The combined organic phases are washed with brine, dried using sodium sulphate and evaporated. The title compound is obtained from the residue by means of flash chromatography (SiO₂ 60F).

### General Method X: (Alcohol Methoxyacetylation)

A solution of 1 mmol of “alcohol” in 13.5 ml of toluene is treated successively at 0° C. with 2.6 mmol of pyridine, 2.4 mmol of methoxyacetyl chloride and 0.1 mmol of 4-dimethylaminopyridine. The ice bath is removed and the reaction mixture is stirred for 2 hours at room temperature. The reaction mixture is poured onto 0.5M HCl and the organic phase is subsequently separated off. The aqueous phase is again extracted with diethyl ether (3×)—the combined organic phases are washed with brine, dried using sodium sulphate and evaporated. The title compound is obtained from the residue by means of flash chromatography (SiO₂ 60F).

### General Method Y (Hydrogenation II)

A solution of 1 mmol of “substrate” in 25 ml of ethanol and ethanolamine (1 mmol) is hydrogenated for 2-5 hours at room temperature in the presence of 600 mg of Pd/C 10% (dry). The reaction mixture is subjected to clarifying filtration and the catalyst is washed with ethanol. The filtrate is evaporated. The residue is treated with 1 M sodium hydrogencarbonate solution and extracted with tert-butyl methyl ether (3×)—the combined organic phases are dried using sodium sulphate and evaporated. The title compound is obtained from the residue by means of flash chromatography (SiO₂ 60F).

### General Method Z (Ester Hydrolysis)

A solution of 1 mmol of “ester” in 3 ml of tetrahydrofuran is treated with 120 mmol of 3M LiOH and stirred at room temperature for 1 hour. The mixture is adjusted to pH2 using 2M HCl and extracted with ethyl acetate (3×). The combined organic phases are dried using sodium sulphate and evaporated. The crude title compound is identified from the residue by means of the Rf value.

### General Method AA: (Pentafluorophenyl Ester Coupling)

A solution of 1 mmol of “pentafluorophenyl ester” in 3 ml of N,N-dimethylformamide is added under a nitrogen atmosphere at 0° C. with stirring to a solution of 1 mmol of “alcohol” or “amine” and 1 mmol of triethylamine in 15 ml of N,N-dimethylformamide. The reaction mixture is stirred at room temperature until the reaction is complete according to thin layer chromatography. It is poured onto pH 3 buffer solution, brought to pH 1 using 1 M HCl and extracted with dichloromethane (2×). The organic phase is dried using sodium sulphate and evaporated. The title compound is obtained from the residue by means of flash chromatography (SiO₂ F60).

### General Method BB: (Halide Nitration)

A mixture of 1 mmol of ‘halide’ and 2.58 mmol of silver nitrate in 5 ml of acetonitrile is heated at 70° C. for 20 minutes in a microwave. Subsequently, it is filtered through hyflo and the resulting solution is evaporated. The title compound is obtained from the residue by means of flash chromatography (SiO₂ F60).

### General Method CC: (Carbonate Formation/Carbamate Formation)

A solution of 1 mmol of “p-nitrophenylcarbonate” in 3 ml of N,N-dimethylformamide is added at 0° C. with stirring to a solution of 1 mmol of “alcohol” or “amine” and 1 mmol of triethylamine in 15 ml of N,N-dimethylformamide unter a nitrogen atmosphere. The reaction mixture is stirred at room temperature until the reaction is complete according to thin layer chromatography. It is poured onto pH 3 buffer solution, brought to pH 1 using 1 M HCl and extracted with dichloromethane (2×). The organic phase is dried using sodium sulphate and evaporated. The title compound is obtained from the residue by means of flash chromatography (SiO₂ F60).

### General Method DD: (4-Nitrophenyl Carbonate Formation)

1 mmol of bis(4-nitrophenyl) carbonate [5070-13-3] and 3 mmol of N,N-dimethyl-aminopyridine in 15 ml of dichloromethane are added at room temperature under a nitrogen atmosphere to a solution of 1 mmol of “alcohol”, and stirred until the reaction is complete according to thin layer chromatography. The mixture is poured onto pH 3 buffer solution, brought to pH 1 using 1 M HCl and extracted with dichloromethane (2×). The organic phase is dried using sodium sulphate and evaporated. The title compound is obtained from the residue by means of flash chromatography (SiO₂ F60).

### Example 1

### (2S,4S,5S,7S)-5-Amino-4-hydroxy-2-isopropyl-7-[4-methoxy-3-(3-methoxypropoxy)-benzyl]-8-methylnonanoic acid (2-nitrooxyethyl)amide

Analogously to method Q, starting from tert-butyl[(1S,2S,4S)-2-hydroxy-1-{(S)-2-[4-methoxy-3-(3-methoxypropoxy)benzyl]-3-methylbutyl}-5-methyl-4-(2-nitrooxyethyl-carbamoyl)hexyl]carbamate the title compound is obtained and identified by means of the Rf value.

The starting material is prepared as follows:

### a) tert-Butyl[(1S,2S,4S)-2-hydroxy-1-{(S)-2-[4-methoxy-3-(3-methoxypropoxy)-benzyl]-3-methylbutyl}-5-methyl-4-(2-nitrooxyethylcarbamoyl)hexyl]carbamate

Analogously to method V, starting from tert-butyl{(1S,3S)-1-((2S,4S)-4-isopropyl-5-oxotetrahydrofuran-2-yl)-3-[4-methoxy-3-(3-methoxypropoxy)benzyl]-4-methyl-pentyl}carbamate [866030-35-5] and 2-nitrooxyethylamine[646-02-6] the title compound is obtained and identified by means of the Rf value.

According to the process described in Example 1, the following compounds are prepared in an analogous manner:

### 4 (2S,4S,5S,7S)-5-Amino-4-hydroxy-2-isopropyl-7-[4-(3-methoxypropyl)-3,4-dihydro-2H-benzo[1,4]oxazin-6-ylmethyl]-8-methylnonanoic acid (2-nitrooxy-ethyl)amide

Starting from tert-butyl{(1S,3S)-1-((2S,4S)-4-isopropyl-5-oxotetrahydrofuran-2-yl)-3-[4-(3-methoxypropyl)-3,4-dihydro-2H-benzo[1,4]oxazin-6-ylmethyl]-4-methylpentyl}-carbamate

The starting materials are prepared as follows:

### a) tert-Butyl{(1S,3S)-1-((2S,4S)-4-isopropyl-5-oxotetrahydrofuran-2-yl)-3-[4-(3-methoxypropyl)-3,4-dihydro-2H-benzo[1,4]oxazin-6-ylmethyl]-4-methylpentyl}-carbamate

Analogously to method R, starting from (3S,5S)-5-{(1S,3S)-1-amino-3-[4-(3-methoxypropyl)-3,4-dihydro-2H-benzo[1,4]oxazin-6-ylmethyl]-4-methylpentyl}3-isopropyldihydrofuran-2-one the title compound is obtained and identified by means of the Rf value.

### b) (3S,5S)-5-{(1S,3S)-1-Amino-3-[4-(3-methoxypropyl)-3,4-dihydro-2H-benzo-[1,4]oxazin-6-ylmethyl]-4-methylpentyl}-3-isoprolyldihydrofuran-2-one

Analogously to method Y, starting from (S)-2-[(S)-2-azido-2-((2S,4S)-4-isopropyl-5-oxotetrahydrofuran-2-yl)ethyl]-1-[4-(3-methoxypropyl)-3,4-dihydro-2H-benzo[1,4]-oxazin-6-yl]-3-methylbutyl methoxyacetate the title compound is obtained as a yellow oil. Rt=4.19 (Gradient I).

### c) (S)-2-[(S)-2-Azido-2-((2S,4S)-4-isopropyl-5-oxotetrahydrofuran-2-yl)ethyl]-1-[4-(3-methoxypropyl)-3,4-dihydro-2H-benzo[1,4]oxazin-6-yl]-3-methylbutyl methoxyacetate

Analogously to method X, starting from (3S,5S)-5-((1S,3S)-1-azido-3-{hydroxy-[4-(3-methoxypropyl)-3,4-dihydro-2H-benzo[1,4]oxazin-6-yl]methyl}-4-methylpentyl)-3-isopropyldihydrofuran-2-one the title compound is obtained as a yellow oil. Rf=0.26 (EtOAc:heptane); Rt=5.44 (Gradient I).

### d) (3S,5S)-5-((1S,3S)-1-Azido-3-{hydroxy-[4-(3-methoxypropyl)-3,4-dihydro-2H-benzo[1,4]oxazin-6-yl]methyl}-4-methylpentyl)-3-isopropyldihydrofuran-2-one

Analogously to method W, starting from 6-bromo-4-(3-methoxypropyl)-3,4-dihydro-2H-benzo[1,4]oxazine[865156-63-4] the title compound is obtained as a beige oil. Rt=5.20 (Gradient1).

### 7 (2S,4S,5S,7S)-5-Amino-4-hydroxy-2-isopropyl-7-[3-(3-methoxypropyl)benzo-furan-5-ylmethyl]-8-methylnonanoic acid (2-nitrooxyethyl)amide

Starting from tert-butyl{(1S,3S)-1-((2S,4S)-4-isopropyl-5-oxotetrahydrofuran-2-yl)-3-[3-(3-methoxypropyl)benzofuran-5-ylmethyl]-4-methylpentyl}carbamate

The starting material is prepared as follows:

### a) tert-Butyl{(1S,3S)-1-((2S,4S)-4-isopropyl-5-oxotetrahydrofuran-2-yl)-3-[3-(3-methoxypropyl)benzofuran-5-ylmethyl]-4-methylpentyl}carbamate

The title compound is prepared from 5-bromo-3-(3-methoxypropyl)benzofuran (*) analogously to the process described in Example 4a-d.

### 10 (2S,4S,5S,7S)-5-Amino-4-hydroxy-2-isopropyl-7-[1-(3-methoxypropyl)-3-methyl-1H-indazol-6-ylmethyl]-8-methylnonanoic acid (2-nitrooxyethyl)amide

Starting from tert-butyl{(1S,3S)-1-((2S,4S)-4-isopropyl-5-oxotetrahydrofuran-2-yl)-3-[1-(3-methoxypropyl)-3-methyl-1H-indazol-6-ylmethyl]-4-methylpentyl}carbamate.

The starting material is prepared as follows:

### a) tert-Butyl{(1S,3S)-1-((2S,4S)-4-isopropyl-5-oxotetrahydrofuran-2-yl)-3-[1-(3-methoxypropyl)-3-methyl-1H-indazol-6-ylmethyl]-4-methylpentyl}carbamate

The title compound is prepared from 6-bromo-1-(3-methoxypropyl)-3-methyl-1H-indazole (*) analogously to the process described in Example 4a-d.

### 13 (2S,4S,5S,7S)-5-Amino-4-hydroxy-2-isopropyl-7-[1-(3-methoxypropyl)-3-methyl-1H-indol-6-ylmethyl]-8-methylnonanoic acid (2-nitrooxyethyl)amide

Starting from tert-butyl{(1S,3S)-1-((2S,4S)-4-isopropyl-5-oxotetrahydrofuran-2-yl)-3-[1-(3-methoxypropyl)-3-methyl-1H-indol-6-ylmethyl]-4-methylpentyl}carbamate

The starting material is prepared as follows:

### a) tert-Butyl{(1S,3S)-1-((2S,4S)-4-isopropyl-5-oxotetrahydrofuran-2-yl)-3-[1-(3-methoxypropyl)-3-methyl-1H-indol-6-ylmethyl]-4-methylpentyl}carbamate

The title compound is prepared from 6-bromo-1-(3-methoxypropyl)-3-methyl-1H-indole (*) analogously to the process described in Example 4a-d.

### 16 (2S,4S,5S,7S)-5-Amino-4-hydroxy-2-isopropyl-7-[6-methoxy-5-(4-methoxy-butyl pyridin-3-ylmethyl]-8-methylnonanoic acid (2-nitrooxyethyl)amide

Starting from tert-butyl{(1S,3S)-1-((2S,4S)-4-isopropyl-5-oxotetrahydrofuran-2-yl)-3-[6-methoxy-5-(4-methoxybutyl)pyridin-3-ylmethyl]-4-methylpentyl}carbamate

The starting material is prepared as follows:

### a) tert-Butyl{(1S,3S)-1-((2S,4S)-4-isopropyl-5-oxotetrahydrofuran-2-yl)-3-[6-methoxy-5-(4-methoxybutyl)pyridin-3-ylmethyl]-4-methylpentyl}carbamate

The title compound is prepared from 5-bromo-2-methoxy-3-(4-methoxybutyl)pyridine (*) analogously to the process described in Example 4a-d.

### 19 (2S,4S,5S,7S)-5-Amino-4-hydroxy-2-isopropyl-7-[6-methoxy-5-(3-methoxy-propoxy)pyridin-3-ylmethyl]-8-methylnonanoic acid (2-nitrooxyethyl)amide

Starting from tert-butyl{(1S,3S)-1-((2S,4S)-4-isopropyl-5-oxotetrahydrofuran-2-yl)-3-[6-methoxy-5-(3-methoxypropoxy)pyridin-3-ylmethyl]-4-methylpentyl}carbamate

The starting material is prepared as follows:

### a) tert-Butyl {(1S,3S)-1-((2S,4S)-4-isopropyl-5-oxotetrahydrofuran-2-yl)-3-[6-methoxy-5-(3-methoxypropoxy)pyridin-3-ylmethyl]-4-methylpentyl}carbamate

The title compound is prepared from 5-bromo-2-methoxy-3-(3-methoxypropoxy)-pyridine (*) analogously to the process described in Example 4a-d.

### 22 (2S,4S,5S,7S)-5-Amino-4-hydroxy-2-isopropyl-7-[3-(3-methoxypropyl)-1-methyl-1H-indol-5-ylmethyl]-8-methylnonanoic acid (2-nitrooxyethyl)amide

Starting from tert-butyl{(1S,3S)-1-((2S,4S)-4-isopropyl-5-oxotetrahydrofuran-2-yl)-3-[6-methoxy-5-(3-methoxypropoxy)pyridin-3-ylmethyl]-4-methylpentyl}carbamate

The starting material is prepared as follows:

### a) tert-Butyl{(1S,3S)-1-((2S,4S)-4-isopropyl-5-oxotetrahydrofuran-2-yl)-3-[3-(3-methoxypropyl)-1-methyl-1H-indol-5-ylmethyl]-4-methylpentyl}carbamate

The title compound is prepared from 5-bromo-3-(3-methoxypropyl)-1-methyl-1H-indole (*) analogously to the process described in Example 4a-d.

### 25 (2S,4S,5S,7S)-5-Amino-4-hydroxy-2-isopropyl-7-[3-(3-methoxypropyl)-1-methyl-1H-indazol-5-ylmethyl]-8-methylnonanoic acid (2-nitrooxyethyl)amide

Starting from tert-butyl{(1S,3S)-1-((2S,4S)-4-isopropyl-5-oxotetrahydrofuran-2-yl)-3-[3-(3-methoxypropyl)-1-methyl-1H-indazol-5-ylmethyl]-4-methylpentyl}carbamate

The starting material is prepared as follows:

### a) tert-Butyl{(1S,3S)-1-((2S,4S)-4-isopropyl-5-oxotetrahydrofuran-2-yl)-3-[3-(3-methoxypropyl)-1-methyl-1H-indazol-5-ylmethyl]-4-methylpentyl}carbamate

The title compound is prepared from 5-bromo-3-(3-methoxypropyl)-1-methyl-1H-indazole (*) analogously to the process described in Example 4a-d.

(*) The preparation of the abovementioned “heteroaryl bromides” is described on pages 20-39 of WO 2005/090305, which are hereby implemented.

### Example 2

### (2S,4S ,5S,7S)-5-Amino-4-hydroxy-2-isopropyl-7-[4-methoxy-3-(3-methoxypropoxy)-benzyl]-8-methylnonanoic acid (4-nitrooxycyclohexyl)amide

Analogously to method Q, the title compound is obtained from tert-butyl[(1S,2S,4S)-2-hydroxy-1-{(S)-2-[4-methoxy-3-(3-methoxypropoxy)benzyl]-3-methylbutyl}-5-methyl-4-(4-nitrooxycyclohexylcarbamoyl)hexyl]carbamate and identified by means of the Rf value.

The starting material is prepared as follows:

### a) tert-Butyl[(1S,2S,4S)-2-hydroxy-1-[(S)-2-[4-methoxy-3-(3-methoxypropoxy)-benzyl]-3-methylbutyl]-5-methyl-4-(4-nitrooxycyclohexylcarbamoyl)hexyl]-carbamate

Analogously to method V, the title compound is obtained from tert-butyl{(1S,3S)-1-((2S,4S)-4-isopropyl-5-oxotetrahydrofuran-2-yl)-3-[4-methoxy-3-(3-methoxypropoxy)-benzyl]-4-methylpentyl}carbamate[866030-35-5] and trans-4-nitrooxycyclohexylamine ([137214-41-6] and identified by means of the Rf value.

According to the process described in Example 2, the following compounds are prepared in an analogous manner:

### 5 (2S,4S,5S,7S)-5-Amino-4-hydroxy-2-isopropyl-7-[4-(3-methoxypropyl)-3,4-dihydro-2H-benzo[1,4]oxazin-6-ylmethyl]-8-methylnonanoic acid (4-nitrooxycyclohexyl)amide

Starting from tert-butyl{(1S,3S)-1-((2S,4S)-4-isopropyl-5-oxotetrahydrofuran-2-yl)-3-[4-(3-methoxypropyl)-3,4-dihydro-2H-benzo[1,4]oxazin-6-ylmethyl]-4-methylpentyl}carbamate (Example 4a)

### 8 (2S,4S,5S,7S)-5-Amino-4-hydroxy-2-isopropyl-7-[3-(3-methoxypropyl)-benzofuran-5-ylmethyl]-8-methylnonanoic acid (4-nitrooxycyclohexyl)amide

Starting from tert-butyl{(1S,3S)-1-((2S,4S)-4-isopropyl-5-oxotetrahydrofuran-2-yl)-3-[3-(3-methoxypropyl)benzofuran-5-ylmethyl]-4-methylpentyl}carbamate (Example 7a)

### 11 (2S,4S,5S,7S)-5-Amino-4-hydroxy-2-isopropyl-7-[1-(3-methoxypropyl)-3-methyl-1H-indazol-6-ylmethyl]-8-methylnonanoic acid (4-nitrooxycyclohexyl)-amide

Starting from tert-butyl{(1S,3S)-1-((2S,4S)-4-isopropyl-5-oxotetrahydrofuran-2-yl)-3-[1-(3-methoxypropyl)-3-methyl-1H-indazol-6-ylmethyl]-4-methylpentyl}carbamate (Example 10a)

### 14 (2S,4S,5S,7S)-5-Amino-4-hydroxy-2-isopropyl-7-[1-(3-methoxypropyl)-3-methyl-1H-indol-6-ylmethyl]-8-methylnonanoic acid (4-nitrooxycyclohexyl)-amide

Starting from tert-butyl{(1S,3S)-1-((2S,4S)-4-isopropyl-5-oxotetrahydrofuran-2-yl)-3-[1-(3-methoxypropyl)-3-methyl-1H-indol-6-ylmethyl]-4-methylpentyl}carbamate (Example 13a)

### 17 (2S,4S,5S,7S)-5-Amino-4-hydroxy-2-isopropyl-7-[6-methoxy-5-(4-methoxy-butyl)pyridin-3-ylmethyl]-8-methylnonanoic acid (4-nitrooxycyclohexyl)amide

Starting from tert-butyl{(1S,3S)-1-((2S,4S)-4-isopropyl-5-oxotetrahydrofuran-2-yl)-3-[6-methoxy-5-(4-methoxybutyl)pyridin-3-ylmethyl]-4-methylpentyl}carbamate (Example 16a)

### 20 (2S,4S,5S,7S)-5-Amino-4-hydroxy-2-isopropyl-7-[6-methoxy-5-(3-methoxy-propoxy)pyridin-3-ylmethyl]-8-methylnonanoic acid (4-nitrooxycyclohexyl)amide

Starting from tert-butyl{(1S,3S)-1-((2S,4S)-4-isopropyl-5-oxotetrahydrofuran-2-yl)-3-[6-methoxy-5-(3-methoxypropoxy)pyridin-3-ylmethyl]-4-methylpentyl}carbamate (Example 19a)

### 23 (2S,4S,5S,7S)-5-Amino-4-hydroxy-2-isopropyl-7-[3-(3-methoxypropyl)-1-methyl-1H-indol-5-ylmethyl]-8-methylnonanoic acid (4-nitrooxycyclohexyl)-amide

Starting from tert-butyl{(1S,3S)-1-((2S,4S)-4-isopropyl-5-oxotetrahydrofuran-2-yl)-3-[6-methoxy-5-(3-methoxypropoxy)pyridin-3-ylmethyl]-4-methylpentyl}carbamate (Example 22a)

### 26 (2S,4S,5S,7S)-5-Amino-4-hydroxy-2-isopropyl-7-[3-(3-methoxypropyl)-1-methyl-1H-indazol-5-ylmethyl]-8-methylnonanoic acid (4-nitrooxycyclohexyl)-amide

Starting from tert-butyl{(1S,3S)-1-((2S,4S)-4-isopropyl-5-oxotetrahydrofuran-2-yl)-3-[3-(3-methoxypropyl)-1-methyl-1H-indazol-5-ylmethyl]-4-methylpentyl}carbamate (Example 25a)

### Example 3

### (2S,4S,5S,7S)-5-Amino-4-hydroxy-2-isopropyl-7-[4-methoxy-3-(3-methoxypropoxy)-benzyl]-8-methylnonanoic acid [1-(2-nitrooxyacetyl)piperidin-4-yl]amide

Analogously to method Q, the title compound is obtained from tert-butyl{(1S,2S,4S)-2-hydroxy-1-{(S)-2-[4-methoxy-3-(3-methoxypropoxy)benzyl]-3-methylbutyl}-5-methyl-4-[1-(2-nitrooxyacetyl)piperidin-4-ylcarbamoyl]hexyl}carbamate and identified by means of the Rf value.

The starting materials are prepared as follows:

### a) tert-Butyl{(1S,2S,4S)-2-hydroxy-1-{(S)-2-[4-methoxy-3-(3-methoxylpropoxy)-benzyl]-3-methylbutyl}-5-methyl-4-[1-(2-nitrooxyacetyl)piperidin-4-ylcarbamoyl]-hexyl}carbamate

Analogously to method V, the title compound is obtained from tert-butyl{(1S,3S)-1-((2S,4S)-4-isopropyl-5-oxotetrahydrofuran-2-yl)-3-[4-methoxy-3-(3-methoxypropoxy)-benzyl]-4-methylpentyl}carbamate[866030-35-5] and 1-(4-aminopiperidin-1-yl)-2-nitrooxyethanone and identified by means of the Rf value.

### b) 1-(4-Aminopiperidin-1-yl)-2-nitrooxyethanone

Analogously to method Q, the title compound is obtained from tert-butyl[1-(2-nitrooxyacetyl)piperidin-4-yl]carbamate and identified by means of the Rf value.

### c) tert-Butyl[1-(2-nitrooxyacetyl)piperidin-4-yl]carbamate

Analogously to method U, the title compound is obtained from tert-butyl piperidin-4-ylcarbamate [73874-95-O] and nitrooxyacetic acid [17711-53-4] and identified by means of the Rf value.

According to the process described in Example 3, the following compounds are obtained in an analogous manner:

### 6 (2S,4S,5S,7S)-5-Amino-4-hydroxy-2-isopropyl-7-[4-(3-methoxypropyl)-3,4-dihydro-2H-benzo[1,4]oxazin-6-ylmethyl]-8-methylnonanoic acid [1-(2-nitro-oxyacetyl)piperidin-4-yl]amide

Starting from tert-butyl{(1S,3S)-1-((2S,4S)-4-isopropyl-5-oxotetrahydrofuran-2-yl)-3-[4-(3-methoxypropyl)-3,4-dihydro-2H-benzo[1,4]oxazin-6-ylmethyl]-4-methylpentyl}-carbamate (Example 4a)

### 9 (2S,4S,5S,7S)-5-Amino-4-hydroxy-2-isopropyl-7-[3-(3-methoxypropyl)benzo-furan-5-ylmethyl]-8-methylnonanoic acid [1-(2-nitrooxyacetyl)piperidin-4-yl]amide

Starting from tert-butyl{(1S,3S)-1-((2S,4S)-4-isopropyl-5-oxotetrahydrofuran-2-yl)-3-[3-(3-methoxypropyl)benzofuran-5-ylmethyl]-4-methylpentyl}carbamate (Example 7a)

### 12 (2S,4S,5S,7S)-5-Amino-4-hydroxy-2-isopropyl-7-[1-(3-methoxypropyl)-3-methyl-1H-indazol-6-ylmethyl]-8-methylnonanoic acid [1-(2-nitrooxyacetyl)-Piperidin-4-yl]amide

Starting from tert-butyl{(1S,3S)-1-((2S,4S)-4-isopropyl-5-oxotetrahydrofuran-2-yl)-3-[1-(3-methoxypropyl)-3-methyl-1H-indazol-6-ylmethyl]-4-methylpentyl}carbamate (Example 10a)

### 15 (2S,4S,5S,7S)-5-Amino-4-hydroxy-2-isopropyl-7-[1-(3-methoxypropyl)-3-methyl-1H-indol-6-ylmethyl]-8-methylnonanoic acid [1-(2-nitrooxyacetyl)-piperidin-4-yl]amide

Starting from tert-butyl{(1S,3S)-1-((2S,4S)-4-isopropyl-5-oxotetrahydrofuran-2-yl)-3-[1-(3-methoxypropyl)-3-methyl-1H-indol-6-ylmethyl]-4-methylpentyl}carbamate (Example 13a)

### 18 (2S,4S,5S,7S)-5-Amino-4-hydroxy-2-isopropyl-7-[6-methoxy-5-(4-methoxy-butyl)pyridin-3-ylmethyl]-8-methylnonanoic acid [1-(2-nitrooxyacetyl)piperidin-4-yl]amide

Starting from tert-butyl{(1S,3S)-1-((2S,4S)-4-isopropyl-5-oxotetrahydrofuran-2-yl)-3-[6-methoxy-5-(4-methoxybutyl)pyridin-3-ylmethyl]-4-methylpentyl}carbamate (Example 16a)

### 21 (2S,4S,5S,7S)-5-Amino-4-hydroxy-2-isopropyl-7-[6-methoxy-5-(3-methoxy-propoxy)pyridin-3-ylmethyl]-8-methylnonanoic acid [1-(2-nitrooxyacetyl)-piperidin-4-yl]amide

Starting from tert-butyl{(1S,3S)-1-((2S ,4S)-4-isopropyl-5-oxotetrahydrofuran-2-yl)-3-[6-methoxy-5-(3-methoxypropoxy)pyridin-3-ylmethyl]-4-methylpentyl}carbamate (Example 19a)

### 24 (2S,4S,5S,7S)-5-Amino-4-hydroxy-2-isopropyl-7-[3-(3-methoxypropyl)-1-methyl-1H-indol-5-ylmethyl]-8-methylnonanoic acid [1-(2-nitrooxyacetyl)-piperidin-4-yl]amide

Starting from tert-butyl{(1S,3S)-1-((2S ,4S)-4-isopropyl-5-oxotetrahydrofuran-2-yl)-3-[3-(3-methoxypropyl)-1-methyl-1H-indol-5-ylmethyl]-4-methylpentyl}carbamate (Example 22a)

### 27 (2S,4S,5S,7S)-5-Amino-4-hydroxy-2-isopropyl-7-[3-(3-methoxypropyl)-1-methyl-1H-indazol-5-ylmethyl]-8-methylnonanoic acid [1-(2-nitrooxyacetyl)-piperidin-4-yl]amide

Starting from tert-butyl{(1S,3S)-1-((2S,4S)-4-isopropyl-5-oxotetrahydrofuran-2-yl)-3-[3-(3-methoxypropyl)-1-methyl-1H-indazol-5-ylmethyl]-4-methylpentyl}carbamate (Example 25a)

### Example 28

### N-{(2S,3S ,5S)-3-Amino-2-hydroxy-5-[4-methoxy-3-(3-methoxypropoxy)benzyl]-6-methylheptyl}-2-(4-nitrooxycyclohexyl)isobutyramide

Analogously to method Q, the title compound is obtained from tert-butyl{(1S,3S)-1-{(S)-1-hydroxy-2-[2-methyl-2-(4-nitrooxycyclohexyl)propionylamino]ethyl}3-[4-methoxy-3-(3-methoxypropoxy)benzyl]-4-methylpentyl}carbamate and identified by means of the Rf value.

The starting materials are prepared as follows:

### a) tert-Butyl {(1S,3S)-1-{(S)-1-hydroxy-2-[2-methyl-2-(4-nitrooxycyclohexyl)-propionylamino]ethyl}-3-[4-methoxy-3-(3-methoxypropoxy)benzyl]-4-methylpentyl}carbamate

Analogously to method U, the title compound is obtained from tert-butyl{(1S,3S)-1-((S)-2-amino-1-hydroxyethyl)-3-[4-methoxy-3-(3-methoxypropoxy)benzyl]-4-methyl-pentyl}carbamate [861901-11-3] and trans-2-methyl-2-(4-nitrooxycyclohexyl)-propionic acid and identified by means of the Rf value.

### b) trans-2-Methyl-2-(4-nitrooxycyclohexyl)propionic acid

Analogously to method Z, the title compound is obtained from methyl trans-2-methyl-2-(4-nitrooxycyclohexyl)propionate and identified by means of the Rf value.

### c) Methyl trans-2-methyl-2-(4-nitrooxycyclohexyl)propionate

Analogously to method S, the title compound is obtained from methyl cis-2-(4-methanesulphonyloxycyclohexyl)-2-methylpropionate [865156-96-3] and identified by means of the Rf value.

According to the process described in Example 28, the following compounds are obtained in an analogous manner:

### 30 N-{(2S,4S,5S)-4-Amino-5-hydroxy-2-isopropyl-6-[2-methyl-2-(4-nitrooxy-cyclohexyl)propionylamino]hexyl}-2-(3-methoxypropoxy)benzamide

Starting from tert-butyl((1S,3S)-1-((S)-2-amino-1-hydroxyethyl)-3-{[2-(3-methoxy-propoxy)benzoylamino]methyl}-4-methylpentyl)carbamate [861451-17-4]

### 32 Methyl (1-{(5S,6S)-5-amino-6-hydroxy-3,3-dimethyl-7-[2-methyl-2-(4-nitrooxy-cyclohexyl)propionylamino]heptanoyl}-1,2,3,4-tetrahydroquinolin-3-yl)-carbamate

Starting from methyl [1-((5S,6S)-7-amino-5-tert-butoxycarbonylamino-6-hydroxy-3,3-dimethylheptanoyl)-1,2,3,4-tetrahydroquinolin-3-yl]carbamate [861444-82-8]

### Example 29

### N-{(2S,3S,5S)-3-Amino-2-hydroxy-5-[4-methoxy-3-(3-methoxypropoxy)benzyl]-6-methylheptyl}-2-methyl-2-nitrooxypropionamide

Analogously to method Q, the title compound is obtained from tert-butyl{(1S,3S)-1-[(S)-1-hydroxy-2-(2-methyl-2-nitrooxypropionylamino)ethyl]-3-[4-methoxy-3-(3-methoxy-propoxy)benzyl]-4-methylpentyl}carbamate and identified by means of the Rf value.

The starting material is prepared as follows:

### a) tert-Butyl{(1S,3S)-1-[(S)-1-hydroxy-2-(2-methyl-2-nitrooxypropionylamino)ethyl]-3-[4-methoxy-3-(3-methoxypropoxy)benzyl]-4-methylpentyl}carbamate

Analogously to method U, the title compound is obtained from tert-butyl{(1S,3S)-1-((S)-2-amino-1-hydroxyethyl)-3-[4-methoxy-3-(3-methoxypropoxy)benzyl]-4-methyl-pentyl}carbamate [861901-11-3] and 2-methyl-2-nitrooxypropionic acid [1617-352] and identified by means of the Rf value.

According to the process described in Example 29, the following compounds are prepared in an analogous manner:

### 31 N-[(2S,4S,5S)-4-Amino-5-hydroxy-2-isopropyl-6-(2-methyl-2-nitrooxy-propionylamino)hexyl]-2-(4-methoxybutoxy)benzamide

Starting from tert-butyl((1S,3S)-1-((S)-2-amino-1-hydroxyethyl)-3-{[2-(3-methoxypropoxy)benzoylamino]methyl}-4-methylpentyl)carbamate [861451-17-4]

### 33 Methyl{1-[(5S,6S)-5-amino-6-hydroxy-3,3-dimethyl-7-(2-methyl-2-nitrooxy-propionylamino)heptanoyl]-1,2,3,4-tetrahydroquinolin-3-yl}carbamate

Starting from methyl [1-((5S,6S)-7-amino-5-tert-butoxycarbonylamino-6-hydroxy-3,3-dimethylheptanoyl)-1,2,3,4-tetrahydroquinolin-3-yl]carbamate [861444-82-8]

### Example 34

### 2-{(2S,4S,5S,7S)-5-Amino-4-hydroxy-2-isopropyl-7-[4-methoxy-3-(3-methoxy-propoxy)benzyl]-8-methylnonanoylamino}ethyl 6-nitrooxyhexanoate

Analogously to method Q, the title compound is obtained from 2-{(2S,4S,5S,7S)-5-tert-butoxycarbonylamino-4-hydroxy-2-isopropyl-7-[4-methoxy-3-(3-methoxypropoxy)-benzyl]-8-methylnonanoylamino}ethyl 6-nitrooxyhexanoate and identified by means of the Rf value.

The starting materials are prepared as follows:

### a) 2-{(2S,4S,5S,7S)-5-tert-Butoxycarbonylamino-4-hydroxy-2-isopropyl-7-[4-methoxy-3-(3-methoxypropoxy)benzyl]-8-methylnonanoylamino}ethyl 6-nitrooxyhexanoate

Analogously to method V, the title compound is obtained from tert-butyl{(1S,3S)-1-((2S,4S)-4-isopropyl-5-oxotetrahydrofuran-2-yl)-3-[4-methoxy-3-(3-methoxypropoxy)-benzyl]-4-methylpentyl}carbamate [866030-35-5] and 2-aminoethyl 6-nitrooxy-hexanoate and identified by means of the Rf value.

### b) 2-Aminoethyl 6-nitrooxyhexanoate

Analogously to method Q, the title compound is obtained from 2-tert-butoxycarbonyl-aminoethyl 6-nitrooxyhexanoate and identified by means of the Rf value.

### c) 2-tert-Butoxycarbonylaminoethyl 6-nitrooxyhexanoate

Analogously to method M, the title compound is obtained from tert-butyl (2-hydroxyethyl)carbamate [26690-80-2] and pentafluorophenyl 6-nitrooxyhexanoate and identified by means of the Rf value.

### d) Pentafluorophenyl 6-nitrooxyhexanoate

Analogously to method BB, the title compound is obtained from pentafluorophenyl 6-bromohexanoate [816464-83-2] and identified by means of the Rf value.

According to the process described in Example 34, the following compounds are obtained in an analogous manner:

### 43 4-{(2S,4S,5S,7S)-5-Amino-4-hydroxy-2-isopropyl-7-[4-methoxy-3-(3-methoxy-propoxy)benzyl]-8-methylnonanoylamino}cyclohexyl 1-nitrooxyethyl carbonate

Starting from tert-butyl{(1S,3S)-1-((2S,4S)-4-isopropyl-5-oxotetrahydrofuran-2-yl)-3-[4-methoxy-3-(3-methoxypropoxy)benzyl]-4-methylpentyl}carbamate [866030-35-5] and 4-aminocyclohexyl 1-nitrooxyethyl carbonate

The starting materials are prepared as follows:

### a) 4-Aminocyclohexyl 1-nitrooxyethyl carbonate

Analogously to method Q, the title compound is obtained from 4-tert-butoxycarbonyl-aminocyclohexyl 1-nitrooxyethyl carbonate and identified by means of the Rf value.

### b) 4-tert-Butoxycarbonylaminocyclohexyl 1-nitrooxyethyl carbonate

Analogously to method CC, the title compound is obtained from tert-butyl (4-hydroxy-cyclohexyl)carbamate [11130-06-2] and 1-nitrooxyethyl 4-nitrophenyl carbonate and identified by means of the Rf value.

### c) 1-Nitrooxyethyl 4-nitrophenyl carbonate

Analogously to method BB, the title compound is obtained from 1-chloroethyl 4-nitrophenyl carbonate-[101623-69-2] and identified by means of the Rf value.

### 47 2-(4-{(2S,4S,5S,7S-5-Amino-4-hydroxy-2-isopropyl-7-[4-methoxy-3-(3-methoxypropoxy)benzyl]-8-methylnonanoylamino}piperidin-1-yl)-2-oxoethyl 4-nitrooxymethylbenzoate

Starting from tert-butyl{(1S,3S)-1-((2S,4S)-4-isopropyl-5-oxotetrahydrofuran-2-yl)-3-[4-methoxy-3-(3-methoxypropoxy)benzyl]-4-methylpentyl}carbamate [866030-35-5] and 2-(4-aminopiperidin-1-yl)-2-oxoethyl 4-nitrooxymethylbenzoate.

The starting materials are prepared as follows:

### a) 2-(4-Aminopiperidin-1-yl)-2-oxoethyl 4-nitrooxymethylbenzoate

Analogously to method Q, the title compound is obtained from 2-(4-tert-butoxy-carbonylaminopiperidin-1-yl)-2-oxoethyl 4-nitrooxymethylbenzoate and identified by means of the Rf value.

### b) 2-(4-tert-Butoxycarbonylaminopiperidin-1-yl)-2-oxoethyl 4-nitrooxymethyl-benzoate

Analogously to method AA, the title compound is obtained from tert-butyl [1-(2-hydroxyacetyl)piperidin-4-yl]carbamate [651056-64-3] and pentafluorophenyl 4-nitrooxymethylbenzoate [874446-96-5] and identified by means of the Rf value.

### Example 35

### 4-{(2S,4S,5S,7S)-5-Amino-4-hydroxy-2-isopropyl-7-[4-(3-methoxypropyl)-3,4-dihydro-2H-benzo[1,4]oxazin-6-ylmethyl]-8-methylnonanoylamino}cyclohexyl 5-nitrooxy-pentanoate

Analogously to method Q, the title compound is obtained from 4-{(2S,4S,5S,7S)-5-tert-butoxycarbonylamino-4-hydroxy-2-isopropyl-7-[4-(3-methoxypropyl)-3,4-dihydro-2H-benzo[1,4]oxazin-6-ylmethyl]-8-methylnonanoylamino}cyclohexyl 5-nitrooxy-pentanoate and identified by means of the Rf value.

The starting materials are prepared as follows:

### a) 4-{(2S,4S,5S,7S)-5-tert-Butoxycarbonylamino-4-hydroxy-2-isopropyl-7-[4-(3-methoxypropyl)-3,4-dihydro-2H-benzo[1,4]oxazin-6-ylmethyl]-8-methylnonanoylamino}cyclohexyl 5-nitrooxypentanoate

Analogously to method V, the title compound is obtained from tert-butyl{(1S,3S)-1-((2S,4S)-4-isopropyl-5-oxotetrahydrofuran-2-yl)-3-[4-(3-methoxypropyl)-3,4-dihydro 2H-benzo[1,4]oxazin-6-ylmethyl]-4-methylpentyl}carbamate (Example 4a) and 4-aminocyclohexyl 5-nitrooxypentanoate and identified by means of the Rf value.

### b) 4-Aminocyclohexyl 5-nitrooxypentanoate

Analogously to method Q, the title compound is obtained from 4-tert-butoxycarbonyl-aminocyclohexyl 5-nitrooxypentanoate and identified by means of the Rf value.

### c) 4-tert-Butoxycarbonylaminocyclohexyl 5-nitroxypentanoate

Analogously to method AA, the title compound is obtained from tert-butyl (4-hydroxycyclohexyl)carbamate [11130-06-2] and pentafluorophenyl 5-nitrooxy-pentanoate [874446-94-3] and identified by means of the Rf value.

### Example 36

### 2-(4-{(2S,4S,5S,7S)-5-Amino-4-hydroxy-2-isopropyl-7-[3-(3-methoxypropyl)benzofuran-5-ylmethyl]-8-methylnonanoylamino}piperidin-1-yl)-2-oxoethyl 4-nitrooxybutanoate

Analogously to method Q, the title compound is obtained from 2-(4-{(2S,4S,5S,7S)-5-tert-butoxycarbonylamino-4-hydroxy-2-isopropyl-7-[3-(3-methoxypropyl)benzofuran-5-ylmethyl]-8-methylnonanoylamino}piperidin-1-yl)-2-oxoethyl 4-nitrooxybutanoate and identified by means of the Rf value.

The starting materials are prepared as follows:

### a) 2-(4-{(2S,4S,5S,7S)-5-tert-Butoxycarbonylamino-4-hydroxy-2-isopropyl-7-[3-(3-methoxypropyl)benzofuran-5-ylmethyl]-8-methylnonanoylamino}piperidin-1-yl)-2-oxoethyl 4-nitrooxybutanoate

Analogously to method V, the title compound is obtained from tert-butyl{(1S,3S)-1-((2S,4S)-4-isopropyl-5-oxotetrahydrofuran-2-yl)-3-[3-(3-methoxypropyl)benzofuran-5-ylmethyl]-4-methylpentyl}carbamate (Example 7a) and 2-(4-aminopiperidin-1-yl)-2-oxoethyl 4-nitrooxybutanoate and identified by means of the Rf value.

### b) 2-(4-Aminopiperidin-1-yl)-2-oxoethyl 4-nitrooxybutanoate

Analogously to method Q, the title compound is obtained from 2-(4-tert-butoxy-carbonylaminopiperidin-1-yl)-2-oxoethyl 4-nitrooxybutanoate and identified by means of the Rf value.

### c) 2-(4-tert-Butoxycarbonylaminopiperidin-1-yl)-2-oxoethyl 4-nitrooxybutanoate

Analogously to method M, the title compound is obtained from tert-butyl [1-(2-hydroxyacetyl)piperidin-4-yl]carbamate [651056-64-3] and pentafluorophenyl 4-nitrooxybutanoate [838878-70-9] and identified by means of the Rf value.

### Example 37

### 2-{(2S,4S,5S,7S-5-Amino-4-hydroxy-2-isopropyl-7-[1-(3-methoxypropyl-3-methyl-1H-indazol-6-ylmethyl]-8-methylnonanoylamino}ethyl 4-nitrooxybutyl carbonate

Analogously to method Q, the title compound is obtained from 2-{(2S,4S,5S,7S)-5-tert-butoxycarbonylamino-4-hydroxy-2-isopropyl-7-[1-(3-methoxypropyl)-3-methyl-1H-indazol-6-ylmethyl]-8-methylnonanoylamino}ethyl 4-nitrooxybutyl carbonate and identified by means of the Rf value.

The starting materials are prepared as follows:

### a) 2-{(2S,4S,5S,7S)-5-tert-Butoxycarbonylamino-4-hydroxy-2-isopropyl-7-[1-(3-methoxypropyl)-3-methyl-1H-indazol-6-ylmethyl]-8-methylnonanoylamino}-ethyl 4-nitrooxybutyl carbonate

Analogously to method V, the title compound is obtained from tert-butyl{(1S,3S)-1- ) ((2S,4S)-4-isopropyl-5-oxotetrahydrofuran-2-yl)-3-[1-(3-methoxypropyl)-3-methyl-1H-indazol-6-ylmethyl]-4-methylpentyl}carbamate (Example 10a) and 2-aminoethyl 4-nitrooxybutyl carbonate and identified by means of the Rf value.

### b) 2-Aminoethyl 4-nitrooxybutyl carbonate

Analogously to method Q, the title compound is obtained from 2-tert-butoxycarbonyl-aminoethyl 4-nitrooxybutyl carbonate and identified by means of the Rf value.

### c) 2-tert-Butoxycarbonylaminoethyl 4-nitrooxybutyl carbonate

Analogously to method CC, the title compound is obtained from tert-butyl (2-hydroxy-ethyl)carbamate [26690-80-2] and 4-nitrooxybutyl 4-nitrophenyl carbonate [935472-60-9] and identified by means of the Rf value.

According to the process described in Example 37, the following compounds are prepared in an analogous manner:

### 44 4-{(2S,4S,5S,7S)-5-Amino-4-hydroxy-2-isopropyl-7-[1-(3-methoxypropyl)-3-methyl-1H-indazol-6-ylmethyl]-8-methylnonanoylamino}cyclohexyl 1-nitrooxy-ethyl carbonate

Starting from tert-butyl{(1S,3S)-1-((2S,4S)-4-isopropyl-5-oxotetrahydrofuran-2-yl)-3-[1-(3-methoxypropyl)-3-methyl-1H-indazol-6-ylmethyl]-4-methylpentyl}carbamate (Example 10a) and 4-aminocyclohexyl 1-nitrooxyethyl carbonate (Example 43a).

### 48 2-(4-{(2S,4S,5S,7S)-5-Amino-4-hydroxy-2-isopropyl-7-[1-(3-methoxypropyl)-3-methyl-1H-indazol-6-ylmethyl]-8-methylnonanoylamino}piperidin-1-yl-2-oxo-ethyl 4-nitrooxymethylbenzoate

Starting from tert-butyl{(1S,3S)-1-((2S,4S)-4-isopropyl-5-oxotetrahydrofuran-2-yl)-3-[1-(3-methoxypropyl)-3-methyl-1H-indazol-6-ylmethyl]-4-methylpentyl}carbamate (Example 10a) and 2-(4-aminopiperidin-1-yl)-2-oxoethyl 4-nitrooxymethylbenzoate.

The starting materials are prepared as follows:

### a) 2-(4-Aminopiperidin-1-yl)-2-oxoethyl 4-nitrooxymethylbenzoate

Analogously to method Q, the title compound is obtained from 2-(4-tert-butoxy-carbonylaminopiperidin-1-yl)-2-oxoethyl 4-nitrooxymethylbenzoate and identified by means of the Rf value.

### b) 2-(4-tert-Butoxycarbonylaminopiperidin-1-yl)-2-oxoethyl 4-nitrooxymethyl-benzoate

Analogously to method AA, the title compound is obtained from tert-butyl [1-(2-hydroxyacetyl)piperidin-4-yl]carbamate [651056-64-3] and pentafluorophenyl 4-nitrooxymethylbenzoate [874446-96-5] and identified by means of the Rf value.

### Example 38

### 4-{(2S ,4S,5S,7S)-5-Amino-4-hydroxy-2-isopropyl-7-[1-(3-methoxypropyl)-3-methyl-1H-indol-6-ylmethyl]-8-methylnonanoylamino}cyclohexyl 3-nitrooxypropyl carbonate

Analogously to method Q, the title compound is obtained from 4-{(2S,4S,5S,7S)-5-tert-butoxycarbonylamino-4-hydroxy-2-isopropyl-7-[1-(3-methoxypropyl)-3-methyl-1H-indol-6-ylmethyl]-8-methylnonanoylamino}cyclohexyl 3-nitrooxypropyl carbonate and identified by means of the Rf value.

The starting materials are prepared as follows:

### a) 4-{(2S,4S,5S,7S)-5-tert-Butoxycarbonylamino-4-hydroxy-2-isopropyl-7-[1-(3-methoxypropyl)-3-methyl-1H-indol-6-ylmethyl]-8-methylnonanoylamino}-cyclohexyl 3-nitrooxypropyl carbonate

Analogously to method V, the title compound is obtained from tert-butyl{(1S,3S)-1-((2S,4S)-4-isopropyl-5-oxotetrahydrofuran-2-yl)-3-[1-(3-methoxypropyl)-3-methyl-1H-indol-6-ylmethyl]-4-methylpentyl}carbamate (Example 13a) and 4-aminocyclohexyl 3-nitrooxypropyl carbonate and identified by means of the Rf value.

### b) 4-Aminocyclohexyl 3-nitrooxypropyl carbonate

Analogously to method Q, the title compound is obtained from 4-tert-butoxycarbonyl-aminocyclohexyl 3-nitrooxypropyl carbonate and identified by means of the Rf value.

### c) 4-tert-Butoxycarbonylaminocyclohexyl 3-nitrooxypropyl carbonate

Analogously to method CC, the title compound is obtained from tert-butyl (4-hydroxy-cyclohexyl)carbamate [11130-06-2] and 4-nitrooxypropyl 4-nitrophenyl carbonate and identified by means of the Rf value.

### d) 4-Nitrooxypropyl 4-nitrophenyl carbonate

Analogously to method DD, the title compound is obtained from 3-nitrooxypropan-1-ol [100502-66-7] and identified by means of the Rf value.

According to the process described in Example 38, the following compounds are prepared in an analogous manner:

### 45 2-(4-{(2S,4S,5S,7S)-5-Amino-4-hydroxy-2-isopropyl-7-[1-(3-methoxypropyl)-3-methyl-1H-indol-6-ylmethyl]-8-methylnonanoylamino}piperidin-1-yl)-2-oxoethyl 1-nitrooxyethyl carbonate

Starting from tert-butyl{(1S,3S)-1-((2S,4S)-4-isopropyl-5-oxotetrahydrofuran-2-yl)-3-[1-(3-methoxypropyl)-3-methyl-1H-indol-6-ylmethyl]-4-methylpentyl}carbamate (Example 13a) and 2-(4-aminopiperidin-1-yl)-2-oxoethyl 1-nitrooxyethyl carbonate.

The starting materials are prepared as follows:

### a) 2-(4-Aminopiperidin-1-yl)-2-oxoethyl 1-nitrooxyethyl carbonate

Analogously to method Q, the title compound is obtained from 2-(4-tert-butoxy-carbonylaminopiperidin-1-yl)-2-oxoethyl 1-nitrooxyethyl carbonate and identified by means of the Rf value.

### b) 2-(4-tert-Butoxycarbonylaminopiperidin-1-yl)-2-oxoethyl 1-nitrooxyethyl carbonate

Analogously to method CC, the title compound is obtained from tert-butyl [1-(2-hydroxyacetyl)piperidin-4-yl]carbamate [651056-64-3] and 1-nitrooxyethyl 4-nitrophenyl carbonate (Example 43c) and identified by means of the Rf value.

### Example 39

### 2-(4-{(2S,4S,5S,7S)-5-Amino-4-hydroxy-2-isopropyl-7-[6-methoxy-5-(4-methoxy-butyl)pyridin-3-ylmethyl]-8-methylnonanoylamino}piperidin-1-yl)-2-oxoethyl 2-(2-nitrooxyethoxy)ethyl carbonate

Analogously to method Q, the title compound is obtained from 2-(4-{(2S,4S,5S,7S)-5-tert-butoxycarbonylamino-4-hydroxy-2-isopropyl-7-[6-methoxy-5-(4-methoxybutyl)-pyridin-3-ylmethyl]-8-methylnonanoylamino}piperidin-1-yl)-2-oxoethyl 2-(2-nitrooxy-ethoxy)ethyl carbonate and identified by means of the Rf value.

The starting materials are prepared as follows:

### a) 2-(4-{(2S,4S,5S,7S)-5-tert-Butoxycarbonylamino-4-hydroxy-2-isopropyl-7-[6-methoxy-5-(4-methoxybutyl)pyridin-3-ylmethyl]-8-methylnonanoylamino}-piperidin-1-yl)-2-oxoethyl 2-(2-nitrooxyethoxy)ethyl carbonate

Analogously to method V, the title compound is obtained from tert-butyl{(1S,3S)-1-((2S,4S)-4-isopropyl-5-oxotetrahydrofuran-2-yl)-[6-methoxy-5-(4-methoxybutyl)-pyridin-3-ylmethyl]-4-methylpentyl}carbamate (Example 16a) and 2-(4-aminopiperidin-1-yl)-2-oxoethyl 2-(2-nitrooxyethoxy)ethyl carbonate and identified by means of the Rf value.

### b) 2-(4-Aminopiperidin-1-yl)-2-oxoethyl 2-(2-nitrooxyethoxy)ethyl carbonate

Analogously to method Q, the title compound is obtained from 2-(4-tert-butoxy-carbonylaminopiperidin-1-yl)-2-oxoethyl 2-(2-nitrooxyethoxy)ethyl carbonate and identified by means of the Rf value.

### c) 2-(4-tert-Butoxycarbonylaminopiperidin-1-yl)-2-oxoethyl 2-(2-nitrooxyethoxy)-ethyl carbonate

Analogously to method CC, the title compound is obtained from tert-butyl [1-(2-hydroxyacetyl)piperidin-4-yl]carbamate [651056-64-3] and 2-(2-nitrooxyethoxy)ethyl 4-nitrophenyl carbonate and identified by means of the Rf value.

### d) 2-(2-Nitrooxyethoxy)ethyl 4-nitrophenyl carbonate

Analogously to method DD, the title compound is obtained from 2-(2-nitrooxyethoxy)-ethanol [20633-16-3] and identified by means of the Rf value.

### Example 40

### 2-{(2S,4S ,5S,7S)-5-Amino-4-hydroxy-2-isopropyl-7-[6-methoxy-5-(3-methoxy-propoxy)pyridin-3-ylmethyl]-8-methylnonanoylamino}ethyl 4-nitrooxymethylbenzoate

Analogously to method Q, the title compound is obtained from 2-{(2S,4S,5S,7S)-5-tert-butoxycarbonylamino-4-hydroxy-2-isopropyl-7-[6-methoxy-5-(3-methoxypropoxy)-pyridin-3-ylmethyl]-8-methylnonanoylamino}ethyl 4-nitrooxymethylbenzoate and identified by means of the Rf value.

The starting materials are prepared as follows:

### a) 2-{(2S,4S,5S,7S)-5-tert-Butoxycarbonylamino-4-hydroxy-2-isopropyl-7-[6-methoxy-5-(3-methoxypropoxy)piperidin-3-ylmethyl]-8-methylnonanoylamino}-ethyl 4-nitrooxymethylbenzoate

Analogously to method V, the title compound is obtained from tert-butyl{(1S,3S)-1-((2S ,4S)-4-isopropyl-5-oxotetrahydrofuran-2-yl)-3-[6-methoxy-5-(3-methoxypropoxy)-pyridin-3-ylmethyl]-4-methylpentyl}carbamate (Example 19a) and 2-aminoethyl 4-nitrooxymethylbenzoate and identified by means of the Rf value.

### b) 2-Aminoethyl 4-nitrooxymethylbenzoate

Analogously to method Q, the title compound is obtained from 2-tert-butoxycarbonyl-aminoethyl 4-nitrooxymethylbenzoate and identified by means of the Rf value.

### c) 2-tert-Butoxycarbonylaminoethyl 4-nitrooxymethylbenzoate

Analogously to method M, the title compound is obtained from tert-butyl (2-hydroxy-ethyl)carbamate [26690-80-2] and pentafluorophenyl 4-nitrooxymethylbenzoate [874446-96-5] and identified by means of the Rf value.

According to the process described in Example 40, the following compounds are prepared in an analogous manner:

### 46 4-{(2S,4S,5S,7S)-5-Amino-4-hydroxy-2-isopropyl-7-[6-methoxy-5-(3-methoxy-Propoxy)pyridin-3-ylmethyl]-8-methylnonanoylamino}cyclohexyl 1-nitrooxyethyl carbonate

Starting from tert-butyl{(1S,3S)-1-((2S,4S)-4-isopropyl-5-oxotetrahydrofuran-2-yl)-3-[6-methoxy-5-(3-methoxypropoxy)pyridin-3-ylmethyl]-4-methylpentyl}carbamate (Example 19a) and 4-aminocyclohexyl 1-nitrooxyethylcarbonate (Example 43a).

### Example 41

### 4-{(2S,4S,5S,7S)-5-Amino-4-hydroxy-2-isopropyl-7-[3-(3-methoxypropyl)-1-methyl-1H-indol-5-ylmethyl]-8-methylnonanoylamino}cyclohexyl 4-nitrooxymethylbenzoate

Analogously to method Q, the title compound is obtained from 4-{(2S,4S,5S,7S)-5-tert-butoxycarbonylamino-4-hydroxy-2-isopropyl-7-[3-(3-methoxypropyl)-1-methyl-1H-indol-5-ylmethyl]-8-methylnonanoylamino}cyclohexyl 4-nitrooxymethylbenzoate and identified by means of the Rf value.

The starting materials are prepared as follows:

### a) 4-{(2S,4S,5S,7S)-5-tert-Butoxycarbonylamino-4-hydroxy-2-isopropyl-7-[3-(3-methoxypropyl)-1-methyl-1H-indol-5-ylmethyl]-8-methylnonanoylamino}-cyclohexyl 4-nitrooxymethylbenzoate

Analogously to method V, the title compound is obtained from tert-butyl{(1S,3S)-1-((2S,4S)-4-isopropyl-5-oxotetrahydrofuran-2-yl)-3-[3-(3-methoxypropyl)-1-methyl-1H-indol-5-ylmethyl]-4-methylpentyl}carbamate (Example 22a) and 4-aminocyclohexyl 4-nitrooxymethylbenzoate and identified by means of the Rf value.

### b) 4-Aminocyclohexyl 4-nitrooxymethylbenzoate

Analogously to method Q, the title compound is obtained from 4-tert-butoxycarbonyl-aminocyclohexyl 4-nitrooxymethylbenzoate and identified by means of the Rf value.

### c) 4-tert-Butoxycarbonylaminocyclohexyl 4-nitrooxymethylbenzoate

Analogously to method AA, the title compound is obtained from tert-butyl (4-hydroxy-cyclohexyl)carbamate [111300-06-2] and pentafluorophenyl 4-nitrooxymethyl-benzoate [874446-96-5] and identified by means of the Rf value.

### Example 42

### 2-(4-{(2S,4S,5S,7S)-5-Amino-4-hydroxy-2-isopropyl-7-[3-(3-methoxypropyl)-1-methyl-1H-indazol-5-ylmethyl]-8-methylnonanoylamino}piperidin-1-yl)-2-oxoethyl 3-nitrooxy-phenyl carbonate

Analogously to method Q, the title compound is obtained from 2-(4-{(2S,4S,5S,7S)-5-tert-butoxycarbonylamino-4-hydroxy-2-isopropyl-7-[3-(3-methoxypropyl)-1-methyl-1H-indazol-5-ylmethyl]-8-methylnonanoylamino}piperidin-1-yl)-2-oxoethyl 3-nitrooxymethylphenyl carbonate and identified by means of the Rf value.

The starting materials are prepared as follows:

### a) 2-(4-{(2S,4S,5S,7S)-5-tert-Butoxycarbonylamino-4-hydroxy-2-isopropyl-7-[3-(3-methoxypropyl)-1-methyl-1H-indazol-5-ylmethyl]-8-methylnonanoylamino}-piperidin-1-yl)-2-oxoethyl 3-nitrooxymethylphenyl carbonate

Analogously to method V, the title compound is obtained from tert-butyl{(1S,3S)-1-((2S,4S)-4-isopropyl-5-oxotetrahydrofuran-2-yl)-3-[3-(3-methoxypropyl)-1-methyl-1H-indazol-5-ylmethyl]-4-methylpentyl}carbamate (Example 25a) and 2-(4-amino-piperidin-1-yl)-2-oxoethyl 3-nitrooxymethylphenyl carbonate and identified by means of the Rf value.

### b) 2-(4-Aminopiperidin-1-yl)-2-oxoethyl 3-nitrooxymethylphenyl carbonate

Analogously to method Q, the title compound is obtained from 2-(4-tert-butoxy-carbonylaminopiperidin-1-yl)-2-oxoethyl 3-nitrooxymethylphenyl carbonate and identified by means of the Rf value.

### c) 2-(4-tert-Butoxycarbonylaminopiperidin-1-yl)-2-oxoethyl 3-nitrooxymethyl-phenyl carbonate

Analogously to method CC, the title compound is obtained from tert-butyl [1-(2-hydroxyacetyl)piperidin-4-yl]carbamate [651056-64-3] and (3-nitrooxymethylphenyl) 4-nitrophenyl carbonate [874447-03-7] and identified by means of the Rf value.

1. Compound of the general formula (I) 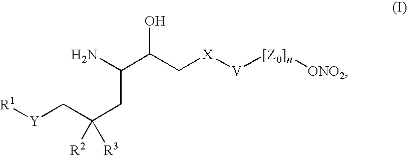
wherein
R¹ is aryl or heterocyclyl, in particular benzoimidazolyl, benzo[1,3]dioxolyl, benzo-furanyl, benzooxazolyl, benzothiazolyl, benzo[b]thienyl, quinazolinyl, quinolyl, quinoxalinyl, 2H-chromenyl, dihydro-2H-benzo[1,4]oxazinyl, dihydro-3H-benzo-[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5-a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetra-hydroimidazo[1,5-a]pyridyl, tetrahydroisoquinolyl, [1,2,3]triazolo[1,5-a]pyridyl or [1,2,4]triazolo[4,3-a]pyridyl, which are substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C -8-alkoxy-C -8-alkoxy, C₁₋₈-alkoxy-C₁₋₈-alkoxy- C -8-alkyl, C₁₋₈-alkoxy-C₁₋₈-alkyl, (N—C₁₋₈-alkoxy)-C₁₋₈-alkylaminocarbonyl-C₁₋₈-alkoxy, (N—C -8-alkoxy)-C₁₋₈-alkylaminocarbonyl-C₁₋₈-alkyl, C₁₋₈-alkoxy-C₁₋₈-alkylcarbamoyl, C₁₋₈-alkoxy-C -8-alkylcarbonyl, C₁₋₈-alkoxy-C₁₋₈-alkylcarbonylamino, 1-C₁₋₈-alkoxy- C₁₋₈-alkylheterocyclyl, C₁₋₈-alkoxyaminocarbonyl-C₁₋₈-alkoxy, C -8-alkoxyamino-carbonyl-C₁₋₈-alkyl, C₁₋₈-alkoxycarbonyl, C₁₋₈-alkoxycarbonyl-C₁₋₈-alkoxy, C₁₋₈-alkoxycarbonyl-C₁₋₈-alkyl, C₁₋₈-alkoxycarbonylamino-C₁₋₈-alkoxy, C₁₋₈-alkoxycarbonyl-amino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonyl-amino-C₁₋₈-alkyl, (N—C₁₋₈-alkyl)-C₁₋₈-alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C₁₋₈-alkyl, C₁₋₈-alkylaminocarbonyl-C₁₋₈-alkoxy, di-C₁₋₈-alkylaminocarbonyl-C₁₋₈-alkoxy, C₁₋₈-alkylaminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkyl-aminocarbonylamino-C₁₋₈-alkyl, C₁₋₈-alkylcarbonylamino, C₁₋₈-alkylcarbonylamino- C₁₋₈-alkoxy, C₁₋₈-alkylcarbonylamino-C₁₋₈-alkyl, C₁₋₈-alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkyl-sulphonylamino-C₁₋₈-alkyl, unalkylated or N-mono- or N,N-di-C₁₋₈-alkylated amino, aryl-C₀₋₈-alkoxy, aryl-C₀₋₈-alkyl, unalkylated or N-mono- or N,N-di-C₁₋₈-alkylated carbamoyl-C₀₋₈-alkoxy, unalkylated or N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halo-C₁₋₈-alkoxy, halo-C₁₋₈-alkyl, heterocyclyl-C₀₋₈-alkoxy, heterocyclyl-C₀₋₈-alkyl, heterocyclylcarbonyl, hydroxy-C -8-alkoxy-C -8-alkoxy, hydroxy-C₁₋₈-alkoxy-C₁₋₈-alkyl, hydroxy-C₁₋₈-alkyl, O-methyl-oximyl-C₁₋₈-alkyl, oxide or oxo;
R² and R³, independently of one another, are hydrogen or C₁₋₆-alkyl or both radicals, together with the carbon atom to which they are bonded, are C₃₋₈-cycloalkyl;
R⁴ is hydrogen or C₁₋₈-alkyl;
V is
-Alk-,
-Alk-O-Alk-,
-aryl-,
-Alk-cycloalkyl-,
-cycloalkyl-,
-cycloalkyl-Alk-,
-Alk-heterocyclyl-,
-heterocyclyl-,
-heterocyclyl-Alk-,
-Alk-heterocyclyl-C(O)-Alk-, or
-heterocyclyl-C(O)-Alk-;
X is —NR⁴—C(O)— or -Alk-C(O)—NR⁴—, wherein Alk designates C₁₋₈-alkylene;
Y is a bond, —C(O)— or —C(O)—NR⁴—;
Z₀ is equal to -Z₁-U-,
wherein Z₁ is —O—C(O)— or —O—C(O)O—;
U is a bivalent radical having the following meaning:
a)
C₁₋₈-alkylene, preferably C₁₋₈-alkylene, unsubstituted or being optionally substituted with one or more of the substituents selected from the group consisting of: halogen, hydroxyl, —ONO₂ or T₀, wherein Tₒ is —OC(O)—(C₁₋₈-alkyl)-ONO₂ or —O—(C₁₋₈-alkyl)-ONO₂;
C₃₋₈-cycloalkylene, the ring being unsubstituted or substituted with side chains T, wherein T is C₁₋₈-alkyl; 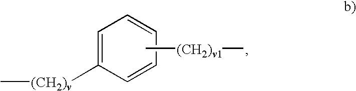
wherein v is an integer from 0 to 20, and v¹ is an integer from 1 to 20; 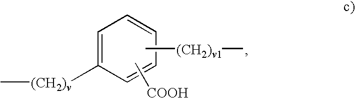
wherein v is an integer from 0 to 20, and v¹ is an integer from 1 to 20; 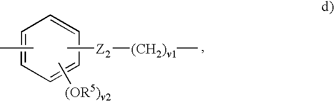
wherein:
v¹ is as defined above and v2 is an integer from 0 to 2;
Z₂=—O—C(O)— or —C(O)—O— and R⁵ is H or CH₃; 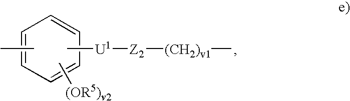
wherein:
v1, v2, R⁵ and Z₂ are as defined above;
U¹ is —CH₂—CH₂— or —CH═CH—(CH₂)ᵥ₂—; 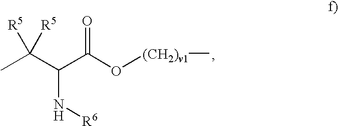
wherein:
v1 and R⁵ are as defined above, R⁶ is H or —C(O)CH₃; 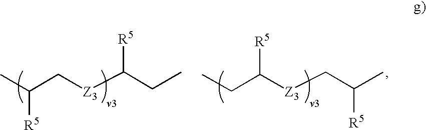
wherein Z₃ is —O— or —S—, v3 is an integer from 1 to 6, preferably from 1 to 4, R⁵ is as defined above; or 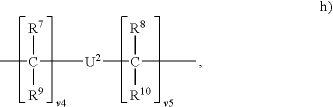
wherein:
v4 is an integer from 0 to 10;
v5 is an integer from 1 to 10;
R⁷, R⁸, R⁹, R¹⁰ are the same or different, and are H or C₁₋₄ alkyl;
U² is a heterocyclic saturated, unsaturated or aromatic 5 or 6 membered ring, containing one or more heteroatoms selected from nitrogen, oxygen and sulphur, and is preferably selected from 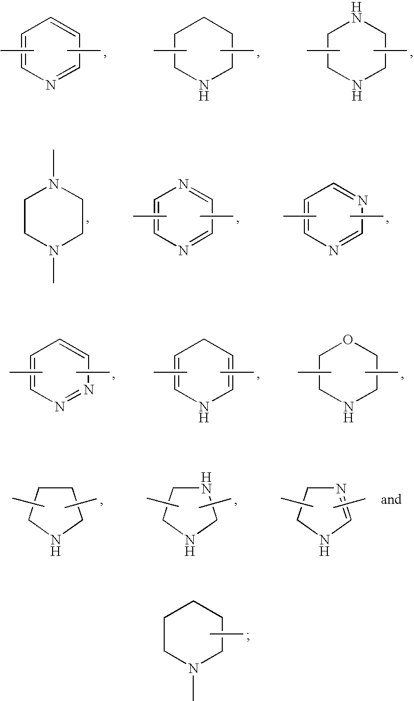
n is 0 or 1;
and their salts, preferably their pharmaceutically usable salts.

2. Compound according to claim 1, wherein R¹ is aryl or heterocyclyl, in particular benzoimidazolyl, benzo[1,3]dioxolyl, benzofuranyl, benzooxazolyl, benzothiazolyl, benzo[b]thienyl, quinazolinyl, quinolyl, quinoxalinyl, 2H-chromenyl, dihydro-2H-benzo[1,4]oxazinyl, dihydro-3H-benzo-[1,4]oxazinyl, dihydro-2H-benzo[1,4]thiazinyl, 2,3-dihydroindolyl, dihydro-1H-pyrido[2,3-b][1,4]oxazinyl, imidazo[1,2-a]pyridyl, imidazo[1,5-a]pyridyl, indazolyl, indolyl, isobenzofuranyl, isoquinolyl, [1,5]naphthyridyl, phenyl, phthalazinyl, pyridyl, pyrimidinyl, 1H-pyrrolo[2,3-b]pyridyl, 1H-pyrrolo[2,3-c]pyridyl, 1H-pyrrolo[3,2-b]pyridyl, tetrahydroquinolyl, tetrahydroquinoxalinyl, tetrahydroimidazo[1,2-a]pyridyl, tetra-hydroimidazo[1,5-a]pyridyl, tetrahydroisoquinolyl, [1,2,3]triazolo[1,5-a]pyridyl or [1,2,4]triazolo[4,3-a]pyridyl, which are substituted by 1-4 acyl-C₁₋₈-alkoxy-C₁₋₈-alkoxy, acyl-C₁₋₈-alkoxy-C₁₋₈-alkyl, (N-acyl)-C₁₋₈-alkoxy-C₁₋₈-alkylamino, C₁₋₈-alkanoyl, C₁₋₈-alkoxy, C₁₋₈-alkoxy-C₁₋₈-alkanoyl, C₁₋₈-alkoxy-C₁₋₈-alkoxy, C₁₋₈-alkoxy-C₁₋₈-alkoxy- C₁₋₈-alkyl, C₁₋₈-alkoxy-C₁₋₈-alkyl, (N—C₁₋₈-alkoxy)-C₁₋₈-alkylaminocarbonyl-C₁₋₈-alkoxy, (N—C₁₋₈-alkoxy)-C₁₋₈-alkylaminocarbonyl-C₁₋₈-alkyl, C₁₋₈-alkoxy-C₁₋₈-alkylcarbamoyl, C₁₋₈-alkoxy-C₁₋₈-alkylcarbonyl, C₁₋₈-alkoxy-C₁₋₈-alkylcarbonylamino, 1-C₁₋₈-alkoxy- C₁₋₈-alkylheterocyclyl, C₁₋₈-alkoxyaminocarbonyl-C₁₋₈-alkoxy, C₁₋₈-alkoxyamino-carbonyl-C₁₋₈-alkyl, C₁₋₈-alkoxycarbonyl, C₁₋₈-alkoxycarbonyl-C₁₋₈-alkoxy, C₁₋₈-alkoxy-carbonyl-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈-alkyl)-C₁₋₈-alkoxy-C₁₋₈-alkylcarbamoyl, (N—C₁₋₈-alkyl)-C₁₋₈-alkoxy-C₁₋₈-alkylcarbonylamino, (N—C₁₋₈-alkyl)-C₁₋₈-alkoxycarbonylamino, (N—C₁₋₈-alkyl)-C₁₋₈-alkylcarbonylamino-C₁₋₈-alkoxy, (N—C₁₋₈-alkyl)-C₁₋₈-alkylcarbonylamino-C₁₋₈-alkyl, (N—C₁₋₈-alkyl)-C₁₋alkylsulphonylamino-C₁₋₈-alkoxy, (N—C₁₋₈-alkyl)-C₁₋₈-alkylsulphonylamino-C₁₋₈-alkyl, C₁₋₈-alkylamidinyl, C₁₋₈-alkylamino-C₁₋₈-alkoxy, di-C₁₋₈-alkylamino-C₁₋₈-alkoxy, C₁₋₈-alkylamino-C₁₋₈-alkyl, di-C₁₋₈-alkylamino-C -8-alkyl, C₁₋₈-alkylaminocarbonyl-C₁₋₈-alkoxy, di-C₁₋₈-alkylaminocarbonyl-C₁₋₈-alkoxy, C₁₋₈-alkyl-aminocarbonyl-C₁₋₈-alkoxy-C₁₋₈-alkyl, C₁₋₈-alkylaminocarbonyl-C₁₋₈-alkyl, di-C₁₋₈-alkylaminocarbonyl-C₁₋₈-alkyl, C₁₋₈-alkylaminocarbonylamino-C₁₋₈-alkoxy, C₁₋₈-alkyl-aminocarbonylamino-C₁₋₈-alkyl, C₁₋₈-alkylcarbonylamino, C₁₋₈-alkylcarbonylamino- C₁₋₈-alkoxy, C₁₋₈-alkylcarbonylamino-C₁₋₈-alkyl, C₁₋alkylcarbonyloxy-C₁₋₈-alkoxy, C₁₋₈-alkylcarbonyloxy-C₁₋₈-alkyl, C₁₋₈-alkylsulphonyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkyl-sulphonylamino-C₁₋₈-alkyl, unalkylated or N-mono- or N,N-di-C₁₋₈-alkylated amino, aryl- C₀₋₈-alkoxy, aryl-C₀₋₈-alkyl, unalkylated or N-mono- or N,N-di-C₁₋₈-alkylated carbamoyl-C₀₋₈-alkoxy, unalkylated or N-mono- or N,N-di-C₁₋₈-alkylated carbamoyl-C₀₋₈-alkyl, carboxy-C₁₋₈-alkoxy, carboxy-C₁₋₈-alkoxy-C₁₋₈-alkyl, carboxy-C₁₋₈-alkyl, cyano, cyano-C₁₋₈-alkoxy, cyano-C₁₋₈-alkyl, C₃₋₈-cycloalkyl-C₁₋₈-alkoxy, C₃₋₈-cycloalkyl-C₁₋₈-alkyl, C₃₋₈-cycloalkylcarbonylamino-C₁₋₈-alkoxy, C₃₋₈-cycloalkylcarbonylamino-C₁₋₈-alkyl, O,N-dimethylhydroxylamino-C₁₋₈-alkyl, halogen, halo-C₁₋₈-alkoxy, halo-C₁₋₈-alkyl, heterocyclyl-C₀₋₈-alkoxy, heterocyclyl-C₀₋₈-alkyl, heterocyclylcarbonyl, hydroxy-C₁₋₈-alkoxy-C₁₋₈-alkoxy, hydroxy-C₁₋₈-alkoxy-C₁₋₈-alkyl, hydroxy-C₁₋₈-alkyl, O-methyl-oximyl-C₁₋₈-alkyl, oxide or oxo;
R² and R³, independently of one another, are hydrogen or C₁₋₆-alkyl or both radicals, together with the carbon atom to which they are bonded, are C₃₋₈-cycloalkyl;
R⁴ is hydrogen or C₁₋₈-alkyl;
V is
-Alk-,
-Alk-O-Alk-,
-aryl-,
-Alk-cycloalkylene-,
-cycloalkylene-,
-cycloalkylene-Alk-,
-Alk-heterocyclyl-,
-heterocyclyl-,
-heterocyclyl-Alk-,
-Alk-heterocyclyl-C(O)-Alk-,
-heterocyclyl-C(O)-Alk-;
X is —NR⁴—C(O)— or -Alk-C(O)—NR⁴—, wherein Alk designates C₁₋₈-alkylene;
Y is a bond, —C(O)— or —C(O)—NR⁴—; and
n is 0.

3. Compound according to claim 1, wherein
R¹ is benzoimidazolyl, 2H-chromenyl, 3,4-dihydro-2H-benzo[1,4]oxazinyl, 1a,7b-dihydro-1H-cyclopropa[c]chromenyl, indazolyl, indolyl, 2,3-dihydro-1H-indolyl, phenyl, pyridyl or 1,1a,2,7b-tetrahydrocyclopropa[c]chromenyl; which are mono- or poly-substituted by C₁₋₈-alkoxy, C₁₋₈-alkoxy-C₁₋₈-alkoxy, C₁₋₈-alkoxy-C₁₋₈-alkoxy-C₁₋₈-alkyl, C₁₋₈-alkoxy-C₁₋₈-alkyl, C₁₋₈-alkoxycarbonylamino-C₁₋₈-alkoxy, C₁₋₈-alkoxycarbonylamino-C₁₋₈-alkyl, C₁₋₈-alkyl, (N—C₁₋₈ alkyl)-C₁₋₈-alkylcarbonylamino-C -8-alkoxy, (N—C₁₋₈-alkyl)-C₁₋₈-alkylcarbonylamino-C₁₋₈-alkyl, (N—C₁₋₈-alkyl)-C₁₋₈-alkylsulphonylamino-C -8-alkoxy, (N—C₁₋₈-alkyl)-C₁₋₈-alkylsulphonylamino-C₁₋₈-alkyl, C₁₋₈-alkylcarbonyl-amino-C₁₋₈-alkoxy, C₁₋₈-alkylcarbonylamino-C₁₋₈-alkyl, C₁₋₈-alkylsulphonyl-C₁₋₈-alkoxy, C₁₋₈-alkylsulphonyl-C₁₋₈-alkyl, C₁₋₈-alkylsulphonylamino-C₁₋₈-alkoxy, C₁₋₈-alkyl-sulphonylamino-C₁₋₈-alkyl, C₃₋₈-cycloalkylcarbonylamino-C₁₋₈-alkoxy, C₃₋₈-cycloalkylcarbonylamino-C₁₋₈-alkyl, halogen, halo-C₁₋₈-alkoxy, halo-C₁₋₈-alkyl or oxide.

4. Compound according to claim 1 of the formula 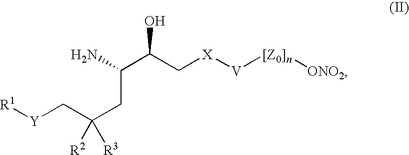
wherein R¹, R², R³, V, X, Y and Z₀ have the meaning as in claim 1.

5-7. (canceled)

8. Method for the prevention, delay of the development, or of the treatment of disease states in a patient which are caused, partly caused by or associated with the renin activity and by a nitrogen monoxide deficiency, or whose state can be improved by inhibition of the renin system and by supply of nitrogen monoxide, which comprises administering a therapeutically efficacious amount of a compound according to claim 1 to a patent in need thereof.

9. Pharmaceutical preparation comprising a compound according to claim 1 and a common additive.

10. Pharmaceutical combination in the form of a preparation or of a kit of individual components consisting of a) a compound according to claim 1, and b) at least one pharmaceutical form whose active agent has a blood pressure-lowering, inotropic, anti-diabetic, lipid-lowering or anti-oxidative effect.

11. Method for the prevention, delay of the development, or of the treatment of disease states in a patient which are caused, partly caused by or associated with the renin activity and by a nitrogen monoxide deficiency, or whose state can be improved by inhibition of the renin system and by supply of nitrogen monoxide, which comprises administering a therapeutically efficacious amount of a compound according to claim 2 to a patent in need thereof.

12. Method for the prevention, delay of the development, or of the treatment of disease states in a patient which are caused, partly caused by or associated with the renin activity and by a nitrogen monoxide deficiency, or whose state can be improved by inhibition of the renin system and by supply of nitrogen monoxide, which comprises administering a therapeutically efficacious amount of a compound according to claim 3 to a patent in need thereof.

13. Method for the prevention, delay of the development, or of the treatment of disease states in a patient which are caused, partly caused by or associated with the renin activity and by a nitrogen monoxide deficiency, or whose state can be improved by inhibition of the renin system and by supply of nitrogen monoxide, which comprises administering a therapeutically efficacious amount of a compound according to claim 4 to a patent in need thereof.

14. Pharmaceutical preparation comprising a compound according to claim 2 and a common additive.

15. Pharmaceutical preparation comprising a compound according to claim 3 and a common additive.

16. Pharmaceutical preparation comprising a compound according to claim 4 and a common additive.

17. Pharmaceutical combination in the form of a preparation or of a kit of individual components consisting of a) a compound according to claim 2, and b) at least one pharmaceutical form whose active agent has a blood pressure-lowering, inotropic, anti-diabetic, lipid-lowering or anti-oxidative effect.

18. Pharmaceutical combination in the form of a preparation or of a kit of individual components consisting of a) a compound according to claim 3, and b) at least one pharmaceutical form whose active agent has a blood pressure-lowering, inotropic, anti-diabetic, lipid-lowering or anti-oxidative effect.

19. Pharmaceutical combination in the form of a preparation or of a kit of individual components consisting of a) a compound according to claim 4, and b) at least one pharmaceutical form whose active agent has a blood pressure-lowering, inotropic, anti-diabetic, lipid-lowering or anti-oxidative effect.
